(19) United States
(12) Patent Application Publication
(10) Pub. No.: US 2023/0018833 A1
(43) Pub. Date: Jan. 19, 2023

(54) GENERATING MULTIMODAL TRAINING DATA COHORTS TAILORED TO SPECIFIC CLINICAL MACHINE LEARNING (ML) MODEL INFERENCING TASKS

(71) Applicant: GE Precision Healthcare LLC, Milwaukee, WI (US)

(72) Inventors: Bipul Das, Chennai (IN); Rakesh Mullick, Bangalore (IN); Utkarsh Agrawal, Bengaluru (IN); KS Shriram, Bangalore (IN); Sohan Ranjan, Bangalore (IN); Tao Tan, Nuenen (NL)

(21) Appl. No.: 17/379,003

(22) Filed: Jul. 19, 2021

(51) Int. Cl. G16H 50/20; G06N 5/04

(52) U.S. Cl. CPC G16H 50/20; G06N 5/04

(57) ABSTRACT

Techniques are described for generating multimodal training data cohorts tailored to specific clinical machine learning (ML) model inferencing tasks. In an embodiment, a method comprises accessing, by a system comprising a processor, multimodal clinical data for a plurality of subjects included in one or more clinical data sources. The method further comprises selecting, by the system, datasets from the multimodal clinical data based on the datasets respectively comprising subsets of the multimodal clinical data that satisfy criteria determined to be relevant to a clinical processing task. The method further comprises generating, by the system, a training data cohort comprising the datasets for training a clinical inferencing model to perform the clinical processing task.

### TECHNICAL FIELD

This application relates to generating multimodal training data cohorts tailored to specific clinical machine learning (ML) model inferencing tasks.

### BACKGROUND

Multimodal machine learning (ML) aims to build models that can process and relate information from multiple modalities. Multimodal ML for automated clinical outcome prediction and diagnosis have recently been gaining attraction for improved clinical inferencing model performance. For example, for prediction of Alzheimer's disease, demographic data with specific lab tests were combined with imaging data as inputs to deep learning models and found improvement over single data source models. Similarly, combining patient demographic information with dermatoscopic images of skin lesions observed a boost in performance as compared to single modality skin cancer models. Other studies have seen similar advantages in a diverse set of medical imaging tasks such as breast cancer prediction, glaucoma classification and detection of microcytic hypochromia.

However, the application of multimodal ML in clinical inferencing tasks brings some unique challenges given the heterogeneity of the data. Prior work has focused on approaches relying on just a few manually selected clinical features from a limited number of modality inputs. Techniques for determining how to efficiently leverage more feature-rich clinical datasets for multimodal clinical inferencing tasks to improve model performance have not yet been explored.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments described herein, systems, computer-implemented methods, apparatus and/or computer program products are provided that facilitate generating multimodal training data cohorts tailored to specific clinical ML model inferencing tasks.

According to an embodiment, a system is provided that comprises a memory that stores computer executable components, and a processor that executes the computer executable components stored in the memory. These computer executable components comprise an access component that accesses multimodal clinical data for a plurality of subjects included in one or more clinical data sources, and a selection component that selects datasets from the multimodal clinical data based on the datasets respectively comprising subsets of the multimodal clinical data that satisfy criteria determined to be relevant to a clinical processing task. The computer executable components further comprise a cohort curation component that generates a training data cohort comprising the datasets for training a clinical inferencing model to perform the clinical processing task.

In one or more embodiments, the multimodal clinical data comprises sets of different types of clinical data for each subject of the plurality of subjects, wherein the subsets respectively comprise clinical data for a different subject of the plurality of subjects, and wherein the selecting comprises selecting the subsets from the sets based on one or more similarity metrics mutual from the different types of clinical data reflecting a consistent anatomy, pathology or diagnosis.

The selection criteria varies for different clinical processing tasks. In some implementations, the selection criteria may be predefined. Additionally, or alternatively, the computer executable components may further comprise a machine learning component that determines the at least some of the criteria using one or more machine learning techniques. For example, in some implementations, the criteria may comprise first criteria and second criteria, and wherein the computer executable components further comprise an extraction component that extracts initial datasets from the multimodal data based on the initial datasets respectively comprising initial multimodal data that that satisfies the first criteria. The machine learning component can further determine the second criteria based on evaluation of the initial datasets using one or more machine learning techniques, and the selection can select the datasets from the initial datasets based on the multimodal data of the datasets respectively satisfying the second criteria.

In some implementations, the computer executable components further comprise an extraction component that extracts diverse features from the multimodal clinical data, wherein the selection component evaluates the diverse features to identify the subsets comprising features of the diverse features that satisfy the criteria, and an importing component that imports the datasets from the one or more clinical data sources based on the subsets comprising the diverse features.

In some implementations in which the datasets comprise medical images, the computer executable components may further comprise an image processing that processes the medical images using one or more pre-processing tasks selected from the group consisting of: image harmonization, image style transfer image resolution augmentation, image data homogenization, and image geometric alignment.

The computer executable components may further comprise a training component that trains the clinical inferencing model to perform the clinical processing task using the training data cohort. The computer executable components may further comprise a learning component that evaluates performance of the clinical inferencing model on new datasets that satisfy the criteria following the training of the clinical inferencing model on the training data cohort and determines one or more features of the new datasets associated with a measure of poor model performance. The computer executable components may further comprise a cohort optimization component that adjusts the criteria based on the one or more features, resulting in updated criteria. With these embodiments, the selection component can further select additional datasets from additional multimodal clinical data based on the additional datasets respectively comprising new subsets of the additional multimodal data that satisfies the updated criteria, wherein the updated criteria requires the new subsets to comprise the one or more features. The curation component can further generate a new training data cohort comprising the additional datasets, and the system can employ the new training data cohort to further train and refine the clinical inferencing model to perform the clinical processing task.

In some embodiments, elements described in the disclosed systems can be embodied in different forms such as a computer-implemented method, a computer program product, or another form.

### DESCRIPTION OF THE DRAWINGS

FIG. 1 illustrates a block diagram of an example, non-limiting system for generating multimodal training data cohorts tailored to specific clinical ML model inferencing tasks in accordance with one or more embodiments of the disclosed subject matter.

FIG. 2 illustrates some example multimodal clinical data in accordance with one or more embodiments of the disclosed subject matter.

FIG. 3 presents an example selection component 112 for selecting clinical ML model input data sets in accordance with one or more embodiments of the disclosed subject matter.

FIG. 4 presents an example cohort curation component in accordance with one or more embodiments of the disclosed subject matter.

FIG. 5 presents a high-level flow diagram of an example process for generating multimodal training data cohorts tailored to specific clinical ML model inferencing tasks in accordance with one or more embodiments of the disclosed subject matter.

FIG. 6 presents a high-level flow diagram of another example process for generating multimodal training data cohorts tailored to specific clinical ML model inferencing tasks in accordance with one or more embodiments of the disclosed subject matter.

FIG. 7 presents a high-level flow diagram of another example process for generating multimodal training data cohorts tailored to specific clinical ML model inferencing tasks in accordance with one or more embodiments of the disclosed subject matter.

FIG. 8 presents a high-level flow diagram of an example process for refining multimodal training data cohort selection for a specific clinical ML model in accordance with one or more embodiments of the disclosed subject matter.

FIG. 9 presents a high-level flow diagram of another example process for generating multimodal training data cohorts tailored to specific clinical ML model inferencing tasks in accordance with one or more embodiments of the disclosed subject matter.

FIG. 10 presents a high-level flow diagram of another example process for refining multimodal training data cohort selection for a specific clinical ML model in accordance with one or more embodiments of the disclosed subject matter.

FIG. 11 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The disclosed subject matter is directed to systems, computer-implemented methods, apparatus and/or computer program products that facilitate generating multimodal training data cohorts tailored to specific clinical ML model inferencing tasks. For a predefined task, the disclosed techniques provide a cohort generation method that down-selects “appropriate” data from a large pool of multimodal clinical data for a variety of different patients with a variety of different clinical profiles. The multimodal clinical data can include medical image data, including medical images captured from different capture modalities and information regarding acquisition or capture parameters. The multimodal clinical data can also include non-imaging data, such as (but is not limited to) radiologists reports, physician notes, laboratory data, physiological parameters, electronic health record (EMR) data, demography data and other non-imaging data.

For example, in some implementations, the input data sets in the training data cohort can include medical image selected based on acquisition related information, scan type, reconstruction kernel size, and other parameters extracted from the medical image data (e.g., including three-dimensional (3D), two-dimensional 2D) and one-dimensional (1D) data parameters) along with non-imaging information from EMR data and clinical findings. This initial set may further be down-selected with other features including geometric signature similarity and biological signature similarity. The down-selected cohort may further be augmented to compensate for differences in resolution, scanner model, and other similar features.

In this regard, the training data cohort selection process uses diverse data features extracted from several different types of clinical data for a same patient to generate a task specific cohort for a given clinical decisioning process. The training data cohort selection process may be partially or fully automated, relying on one or more data selection algorithms. These data selection algorithms may include rule-based algorithms, ML algorithms and/or combinations thereof. Once an initial training data cohort has been generated, the initial training data cohort can be used to train and develop the targeted clinical inferencing model for the predefined task.

The disclosed techniques further provide a learning-based method for continuous data ingestion and model optimization. In this regard, as new multimodal clinical data comes in following model training and deployment, a learning-based method is adopted to select new datasets from the new multimodal clinical data such that the model performance is best improved. This sub-selection process involves tailoring the initial data selection criteria to obtain datasets that are closer to or further from the clinical inferencing model decision boundary. The clinical inferencing model may further be continuously and/or regularly retrained using the new sub-selected datasets to optimize model performance.

The term “cohort” as used herein refers to the entirety of a training dataset selected for training a ML model. As defined herein, a cohort is comprised of a plurality of discrete input data sets for processing by the ML model, wherein each of the input data sets are comprised of data that satisfies defined criteria for inclusion in the cohort. In this regard, because each of the datasets satisfy the defined criteria, each of the data sets are considered similar. The similarity can be defined by one or more parameters of choice depending on the task at hand for cohort creation. These one or more parameters can thus be used as the inclusion or exclusion criteria for selection of the data into the cohort. In various embodiments, the data included in each of the input data sets includes multimodal data (e.g., two or more modality data types) for processing as input into the ML model. Additionally, or alternatively, multimodal data associated with each of the input data sets may be used to facilitate data selection and curation.

The term “multimodal data” is used herein to refer to two or more different types of data. The differentiation factor between the two or more different types of data can vary. For example, the differentiation factor can refer to the medium of the data (e.g., image data, text data, signal data, etc.), the format of the data, the capture modality of the data, the source of the data and so one. In the medical/clinical context, multimodal clinical refers to two or more forms of health-related information that is associated with patient care and/or part of a clinical trial program. Clinical data consist of information ranging from determinants of health and measures of health and health status to documentation of care delivery. Different types of clinical data are captured for a variety of purposes and stored in numerous databases across healthcare systems. Some example types of clinical data that may be included in a pool of multimodal clinical data from which a data cohort may be generated includes (but is not limited to): medical images and associated metadata (e.g., acquisition parameters), radiology reports, clinical laboratory data, patient EHR data, patient physiological data, pharmacy information, pathology reports, hospital admission data, discharge and transfer data, discharge summaries, and progress notes.

The term “clinical inferencing model” is used herein to refer to a ML model configured to perform a clinical decision/processing on clinical data. The clinical decision/processing task can vary. For example, the clinical decision/processing tasks can include classification tasks (e.g., disease classification/diagnosis), disease progression/quantification tasks, organ segmentation tasks, anomaly detection tasks, image reconstruction tasks, and so on. The clinical inferencing models can employ various types of ML algorithms, including (but not limited to): deep learning models, neural network models, deep neural network models (DNNs), convolutional neural network models (CNNs), generative adversarial neural network models (GANs) and the like. The term “multimodal clinical inferencing model” is used herein to refer to a clinical inferencing model adapted to receive and process multimodal clinical data as input.

As used herein, a “medical imaging inferencing model” refers to an image inferencing model that is tailored to perform an image processing/analysis task on one or more medical images. For example, the medical imaging processing/analysis task can include (but is not limited to): disease/condition classification, disease region segmentation, organ segmentation, disease quantification, disease/condition staging, risk prediction, temporal analysis, anomaly detection, anatomical feature characterization, medical image reconstruction, and the like. The terms “medical image inferencing model,” “medical image processing model,” “medical image analysis model,” and the like are used herein interchangeably unless context warrants particular distinction amongst the terms.

The types of medical images processed/analyzed by the medical image inferencing models described herein can include images captured using various types of image capture modalities. For example, the medical images can include (but are not limited to): radiation therapy (RT) images, X-ray (XR) images, digital radiography (DX) X-ray images, X-ray angiography (XA) images, panoramic X-ray (PX) images, computerized tomography (CT) images, mammography (MG) images (including a tomosynthesis device), a magnetic resonance imaging (MRI) images, ultrasound (US) images, color flow doppler (CD) images, position emission tomography (PET) images, single-photon emissions computed tomography (SPECT) images, nuclear medicine (NM) images, and the like. The medical images can also include synthetic versions of native medical images such as synthetic X-ray (SXR) images, modified or enhanced versions of native medical images, augmented versions of native medical images, and the like generated using one or more image processing techniques. The medical imaging processing models disclosed herein can also be configured to process 3D images.

The term “multimodal image data” refers to image data captured of the same subject and anatomical region with two or more different captured modalities and/or acquisition protocol. A “capture modality” as used herein refers to the specific technical mode in which an image or image data is captured using one or more machines or devices. For example, multimodal image data for a particular patient may include an XR imaging study and a CT imaging study captured of the same anatomical region of the patient. In this regard, as applied to medical imaging, different capture modalities can include but are not limited to: a 2D capture modality, a 3D capture modality, an RT capture modality, a XR capture modality, a DX capture modality, a XA capture modality, a PX capture modality a CT, a MG capture modality, a MRI capture modality, a US capture modality, a CD capture modality, a PET capture modality, a SPECT capture modality, a NM capture modality, and the like.

As used herein, a “3D image” refers to digital image data representing an object, space, scene, and the like in three dimensions, which may or may not be displayed on an interface. 3D images described herein can include data representing positions, geometric shapes, curved surfaces, and the like. In an aspect, a computing device, such as a graphic processing unit (GPU) can generate a 3D image based on the data, performable/viewable content in three dimensions. For example, a 3D image can include a collection of points represented by 3D coordinates, such as points in a 3D Euclidean space (e.g., a point cloud). The collection of points can be associated with each other (e.g. connected) by geometric entities. For example, a mesh comprising a series of triangles, lines, curved surfaces (e.g. non-uniform rational basis splines (“NURBS”)), quads, n-grams, or other geometric shapes can connect the collection of points. In an aspect, portions of the mesh can include image data describing texture, color, intensity, and the like.

In various embodiments, captured 2D images (or portions thereof) can be associated with portions of the mesh. A 3D image can thus be generated based on 2D image data, 2D sensory data, sensory data in combination with raw 2D data, 3D spatial data (e.g., spatial depth and distance information), computer generated positional data, and the like. In an aspect, data used to generate 3D images can be collected from 1D and 2D data. Data can also be generated based on computer implemented 3D modeling systems. In some embodiments, a 3D image can be or include a 3D volume image that provides a 3D representation or model of an object or environment generated from a plurality of 2D images captured along different planes. For example, a CT volume image can be or correspond to a 3D representation of an anatomical region of a patient generated/computed from a series of CT scan slices captured along different planes. In this regard, as applied to medical imaging, a 3D image can be or include a 3D volume image of anatomical region of a patient.

In this regard, a 3D medical image refers to a 3D representation of an anatomical region of a patient. In some implementations, a 3D medical image can be captured in 3D directly by the acquisition device and protocol. In other implementations, a 3D medical image can comprise a generated image that was generated from 2D and/or 3D image data captured of the anatomical region of the patient. Some example 3D medical images include 3D volume images generated from CT image data, MRI image data, and US image data.

It is noted that the terms “3D image,” “3D volume image,” “volume image,” “3D model,” “3D object,”, “3D reconstruction,” “3D representation,” “3D rendering,” and the like are employed interchangeably throughout, unless context warrants particular distinctions among the terms. It should be appreciated that such terms can refer to data representing an object, an anatomical region of the body, a space, a scene, and the like in three dimensions, which may or may not be displayed on an interface. The terms “3D data,” can refer to data utilized to generate a 3D image, data describing a 3D image, data describing perspectives or points of view of a 3D image, capture data (e.g. sensory data, images, etc.), meta-data associated with a 3D image, and the like. It is noted that the term a “2D image” as used herein can refer to data representing an object, an anatomical region of the body, a space, a scene, and the like in two dimensions, which may or may not be displayed on an interface.

The term “native” image is used herein to refer to an image in its original capture form and/or its received form prior to processing by the disclosed systems. In this regard, a native 3D image refers to a 3D image in its received state prior to pre-projection processing, transformation processing, projection processing, and post-projection/transformation processing. For example, a native 3D image can include a received 3D volume image, such a s CT volume image. The term “synthetic” image is used herein to distinguish from native images and refers to an image generated or derived from a native image using one or more transformation processing techniques disclosed herein. In various embodiments, a synthetic image refers to a second modality image generated and/or derived from a first modality image. For example, in some embodiments, the second modality image comprises a 2D modality image (e.g., an XR modality) and the first modality image comprises a 3D modality image (e.g., a CT modality).

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

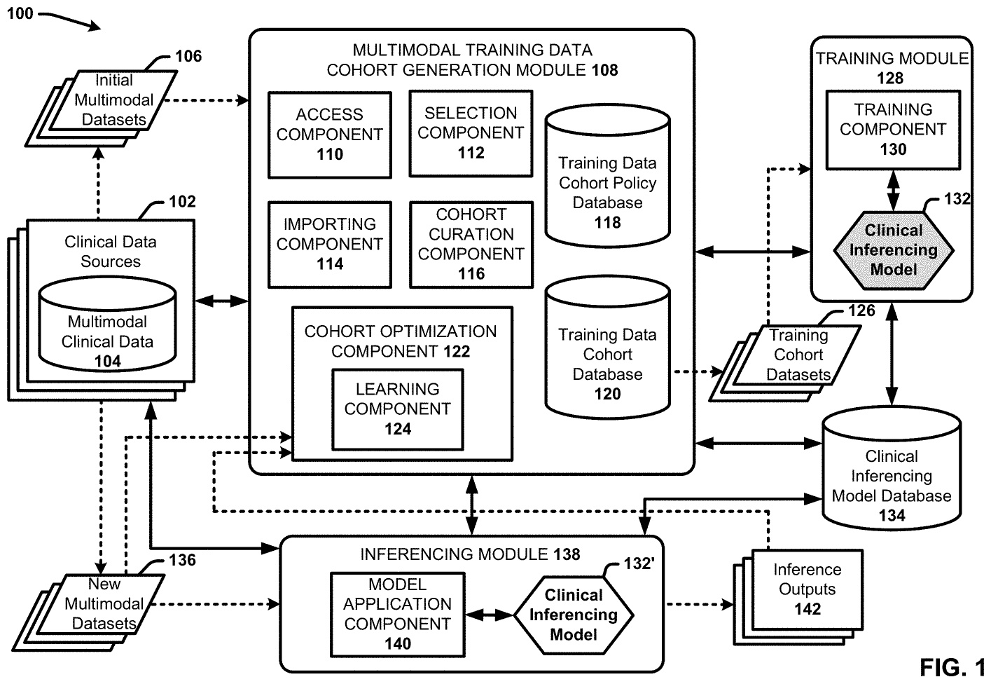

Turning now to the drawings, FIG. 1 illustrates a block diagram of an example, non-limiting system 100 for generating multimodal training data cohorts tailored to specific clinical ML model inferencing tasks in accordance with one or more embodiments of the disclosed subject matter. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

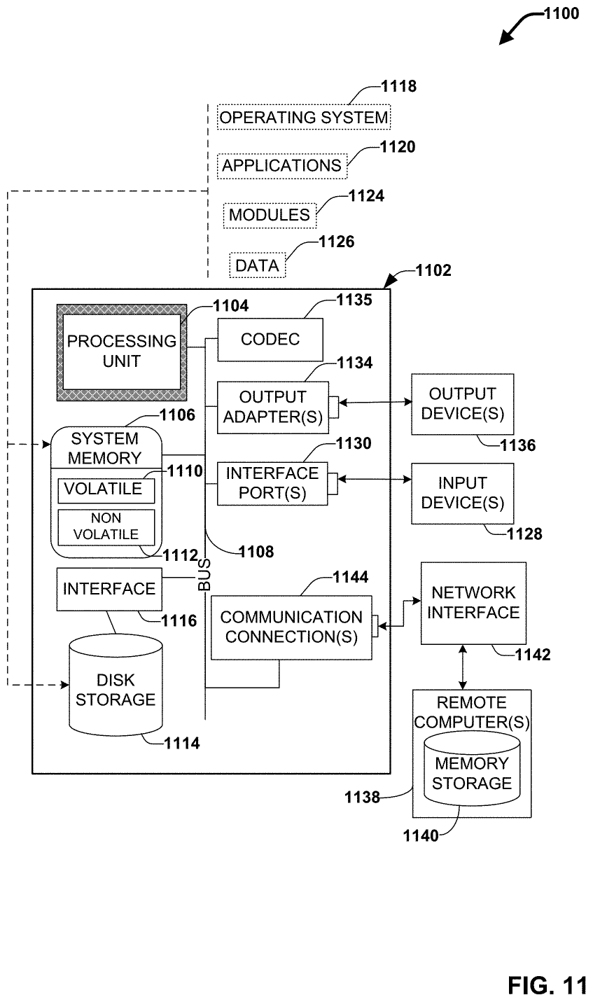

For example, system 100 includes multimodal training data cohort generation module 108, training module 128 and inferencing module 138 which can respectively be and include computer/machine executable components. These computer/machine executable components (and other described herein) can be stored in memory (not shown) associated with the one or more machines (not shown). The memory can further be operatively coupled to at least one processor (not shown), such that the components (e.g., the multimodal training data generation module 108, the training module 128, the inferencing module 138, and other components described herein), can be executed by the at least one processor to perform the operations described. Examples of said and memory and processor as well as other suitable computer or computing-based elements, can be found with reference to FIG. 11, and can be used in connection with implementing one or more of the systems or components shown and described in connection with FIG. 1 or other figures disclosed herein.

System 100 further includes one or more clinical data sources 102 that can collectively provide a pool of multimodal clinical data for a plurality of different patients/subjects for processing by the multimodal training data cohort generation module 108 and the inferencing module 138. In the embodiment shown, this pool of multimodal clinical data is represented by multimodal clinical data 104. For example, the clinical data sources 102 may include various disparate electronic medical databases, systems and/or devices/machines that generate, receive and/or store clinical data for patients/subjects, such as but not limited to, medical imaging data, analysis and reports data, demography data, disease data and the like. These clinical data sources may include for example, picture archiving and communication systems (PACS), electronic medical record (EMR) systems, radiologist reporting systems, laboratory reporting systems, clinical ordering systems, and the like. The clinical data sources 102 may be associated with same or different clinical institutions (e.g., hospitals, hospital systems, clinics, medical facilities, medical imaging facilities, medical laboratory systems, etc.) at various locations worldwide. The multimodal training data cohort generation module 108 can be communicatively and/or operatively connected to the clinical data sources via one or more wired and/or wireless communication networks (e.g., the Internet, an intranet, etc.).

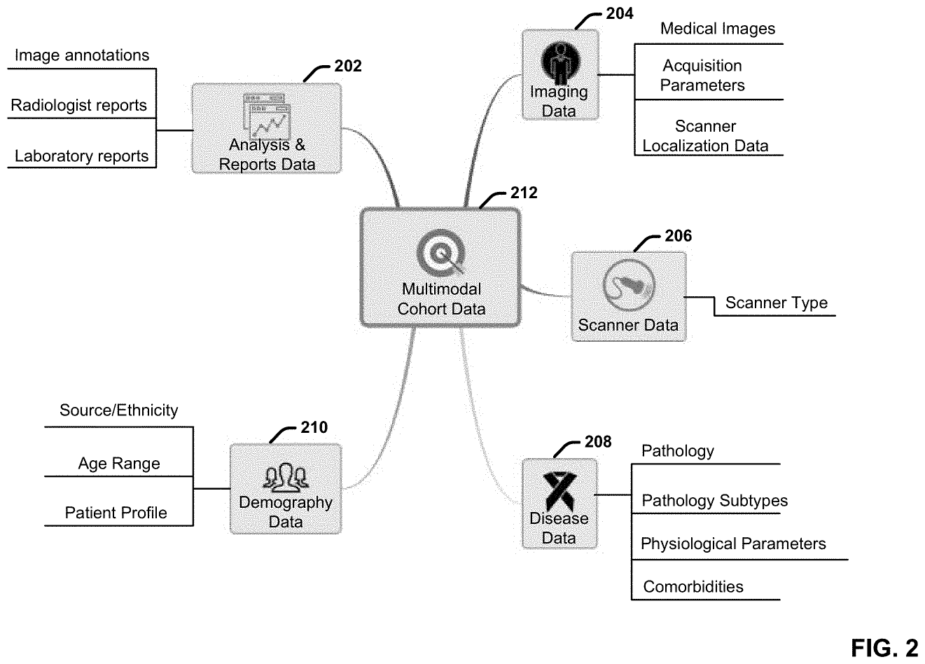

FIG. 2 illustrates some example multimodal data that may be used to generate a multimodal cohort data 212 in accordance with one or more embodiments of the disclosed subject matter. For example, with reference to FIGS. 1 and 2, the variety of multimodal data shown in FIG. 2 can correspond to the pool of multimodal clinical data 104 from which the multimodal training data cohort generation module 108 can selectively generate training data cohorts for tailored to specific clinical inferencing models and/or specific clinical inferencing tasks. This multimodal data can include clinical and non-clinical information for a plethora of diverse patients/subjects with different demography profiles, different pathologies and different medical histories. In the embodiment shown, the multimodal data is grouped into analysis and reports data 202, imaging data 204, scanner data 206, disease data 208 and demography data 210.

The analysis and reports data 202 can include clinical reports and evaluations regarding clinical evaluations of patients, including analysis of imaging and laboratory studies performed for the patients. For example, the analysis and reports data 202 can include radiologist reports, laboratory reports, image annotations and associated metadata. The image annotations may include a variety of annotations applied to medical images for a patient. For example, the image annotations can include mark-ups (e.g., bounding boxes, scan-plane lines, enclosing ellipsoids, and other mark-ups) applied to medical images identifying regions of interest (ROI), segmentation masks, anatomical features, lesions and so on. These annotations can include manually applied annotations as well as machine generated annotations (e.g., generated using one or more medical image inferencing models). Additionally, or alternatively, the annotation data can include information extracted from the applied mark-ups defining the relative position, size, shape, geometry, etc. of the anatomical features defined by the mark-up data.

The imaging data 204 can include medical images/imaging studies captured for patients and other non-imaging parameters associated with medical images/imaging studies (e.g., acquisition protocol information, scanner device information, capture time, etc.). For example, the medical images can include a variety of different medical imaging studies performed for a pool of patients/subjects with a variety of different medical conditions and/or physiological states. In addition to the medical images themselves, the imaging data 204 can also include or otherwise be associated with a variety of rich information regarding the acquisition parameters/protocol and scanner localization. The acquisition parameters/protocol information can vary depending on the modality of the imaging study performed. Some example acquisition parameters/protocol information may include (but is not limited to): contrast or non-contrast, imaging frequency, reconstruction kernel size, imaging slice thickness, CT dose, view of an XR image, MR sequence, capture resolution, voxel size, and study time.

The scanner localization data can include information regarding the position, orientation and field of view of the imaging scanner relative to one or more anatomical features/planes of reference. In some implementations, the scanner localization data may also include information identifying the relative size, shape and/or position of anatomical features and/or scan-planes. For example, the scanner localization data can provide the relative position and dimensions of the bounding box used to define the capture region, the relative position and orientation of the prescription scan-plane used to capture the image data (e.g., relative to the bounding box and/or one or more anatomical landmarks). In some implementations, the scanner localization data may also include one or more low resolution calibration images (e.g., scout images, localizer images, etc.) captured of the patient's region of interest to be scanned prior to capture of subsequent high-resolution (e.g., isotropic) 3D image data 112. The calibration images are generally used to position/align the scanner relative to a desired scan prescription plane for which the 3D image data is captured.

The scanner data 206 can include information regarding the actual imaging machine/scanner used to generate the imaging data 204. For example, the scanner data 206 can include information identifying the scanner type, make, model and so on. The scanner data 206 may also include information regarding the particular imaging site/facility where the imaging study was performed and the operating technician who performed the study.

The disease data 208 can include a variety of rich clinical information associated with the patients regarding their current and/or historical medical condition/status. For example, the disease data 208 can include information regarding a patient's pathology and pathology subtype, physiological parameters, commodities and other tracked information regarding their medical condition/state. The disease data 208 can further be associated with timing information that indicates the patient's tracked physiological state/condition over time. This information can be used to track disease progression, treatment, and correlate physiological condition and status information for a patient at respective points in time when imaging studies were performed, when laboratory studies were performed and so on.

The demography data 210 can include a variety of non-clinical demographic type information associated with the patients. For example, the demography data 210 can include information regarding the patients' source (i.e., home location, treatment location, current location, etc.), ethnicity, age, body mass index (BMI), height/weight, gender and so on. The demography data 210 can also include other relevant information known about the patients included in their patient profiles and/or electronic health records.

With reference to FIGS. 1 and 2, in accordance with system 100, the multimodal training data cohort generation module 108 is configured to access and filter the pool of multimodal clinical data 104 to extract and generate multimodal training data cohorts that are tailored to specific clinical inferencing models. These clinical inferencing models may be stored in one or more clinical inferencing model databases 134 that are accessible to the multimodal training data cohort generation module 108, the training module 128 and the inferencing module 138 via one or more wired or wireless communication networks. The type of the clinical inferencing models can vary. The multimodal training data cohorts generated by the multimodal training data cohort generation module 108 may be stored in the training data cohort database 120. The training module 128 is configured to employ the multimodal training data cohort datasets (training cohort datasets 126) generated for a particular target clinical inferencing model 132 to train and/or develop the particular target clinical inferencing model. Once at least some initial training has been completed, the inferencing module 138 is configured to apply the trained clinical inferencing model 132′ to new multimodal datasets 136 in the field to generate corresponding inference outputs 142. As discussed in greater detail infra, these new multimodal datasets 136 can be selected/extracted from the multimodal clinical data 104 based on the training data cohort selection criteria used to generate the training cohort datasets 126 for the clinical inferencing model.

In the embodiment shown, the clinical inferencing model 132 associated with the training module 128 is distinguished from the clinical inferencing model 132′ associated with the inferencing module 138 to indicate its development status. In this regard, the clinical inferencing model 132 associated with the training module 128 is shown in grey to indicate that it is under training and development, while the clinical inferencing models 132′ associated with the inferencing module 138 is shown in white to indicate it has completed at least initial training and development as is ready for deployment in the field. In this regard, it should be appreciated that the clinical inferencing models 132 and the clinical inferencing models 132′ are the same models. The clinical inferencing model 132/132′ may be stored in a clinical inferencing model database 134 that is accessible to the training module 128, the multimodal training data generation module 108 and/or the inferencing module 138.

The clinical inferencing model 132/132′ depicted in system 100 corresponds to a single clinical inferencing model adapted to perform a specific inferencing task. The clinical inferencing model 132/132′ is referred to as a “target” model to indicate that the training data cohort generation process is tailored for the specific target model and the particular inferencing task the target model is/will be adapted to perform by the training module 128. It should be appreciated however that system 100 can be applied for a variety of different target clinical inferencing models stored in the clinical inferencing model database 134, and the specific data types and data features of the multimodal training data cohort generated for each target clinical inferencing model will be tailored to each target model. In this regard, the multimodal training data cohort selection/preprocessing policy rules and criteria for each target clinical inferencing model may vary. As described in greater detail infra, the selection/preprocessing policy rules and criteria for each target clinical inferencing model can be defined in the training data cohort policy database 118.

In this regard, the particular type and clinical inferencing/processing task of the clinical inferencing model 132/132′ can vary. In various embodiments, the clinical inferencing model 132/132′ includes a model configured to process multimodal clinical input data. For example, the clinical inferencing model 132/132′ can be configured to process a plurality of input features extracted from two or more clinical data objects for a same patient, such as but not limited to, two or more images captured in different modalities (e.g., XR and CT), metadata associated with the images describing acquisition parameters, radiologist reports, laboratory reports, electronic health record information for the patient, and so on. In some embodiments, the clinical inferencing model 132/132 can be or include a medical image inferencing model configured to perform a clinical inferencing task related to a medical condition reflected in one or more medical input image. With these embodiments, each of the cohort multimodal datasets 126 may include input data sets that respectively include at least one input medical image for a patient/subject and each input data set may be associated with a different patient. The multimodal datasets 126 may also include additional data inputs and/or features extracted from other clinical data associated with the same patient/input image (e.g., acquisition parameter data, analysis and reports data, demography data, disease data, etc.).

The clinical inferencing tasks can include tasks related to triage, such as classification of the medical condition, segmentation of a disease region associated with the medical condition, segmentation of an organ associated with the medical condition or the like. For instance, as applied to triage of COVID-19 disease based on chest medical images (e.g., provided in one or more capture modalities), the clinical inferencing models 132/132′ can include a model for classifying the chest images with and without the disease, a model for segmenting the COVID-19 disease region to facilitate further inspection by radiologists, a model for segmenting the entire lung even in the presence of lung consolidation and other abnormalities, and the like. The clinical inferencing tasks can include tasks related to disease quantification, staging and risk prediction. For example, in some implementations, the clinical inferencing model 132/132′ can include a model for computing biomarker metrics such as disease region/total lung region expressed as a ratio in XR images. In another example, the clinical inferencing model 132/132′ can include a model that uses volumetric measures in paired CT and XR image data to build a regression model in XR to obtain volumetric measurements from chest XR images. In another example, clinical inferencing model 132/132′ can include a model that determines whether a patient needs a ventilator or not based on chest XR data combined with other multimodal data inputs using regression analysis when outcomes data is available in addition to the image data for training. In another example, the clinical inferencing model 132/132′ can include a model configured to perform temporal analysis and monitor changes in the disease region over time. It should be appreciated that the different clinical inferencing models described above are merely exemplary and not intended to limit the scope of the disclosed subject matter.

In the embodiment shown, the dashed arrows indicate directional dataflow between respective system components/modules and the solid arrow lines indicate communicative connections between the respective system components/modules. In this regard, the clinical data sources 102, the multimodal training data cohort generation module 108, the training module 128, the clinical inferencing model database 134 and the inferencing module 138 may be communicatively and/or operatively connected to one another via one or more wired or wireless communication networks. However, the deployment architecture of system 100 can vary. For example, in some embodiments, the multimodal training data generation module 108 (and/or one or more components associated therewith), the training module 128, and the inferencing module 138 can be deployed at different computing devices/machines in a distributed computing environment and communicatively coupled via one or more networks (e.g., a wide area network (WAN), a local area network (LAN), or the like). In other embodiments, the respective modules can be deployed at a same computing device in a local deployment architecture. Various alternative deployment architecture variations can also be used.

With reference to the multimodal training data cohort generation module 108, to facilitate generating the multimodal training data cohorts, the multimodal training data cohort generation module 108 can include access component, selection component 112, importing component 114, cohort curation component 116, cohort optimization component 122, training data cohort optimization component 122, training data cohort policy database 118 and training data cohort database 120. These components (and other components described herein) can respectively correspond to computer executable components stored in memory (e.g., system memory 1106 or the like), which when executed by a processor (e.g., processing unit 1104) perform the operations described.

The access component 110 can access the multimodal clinical data 104 provided by clinical data sources 102 to facilitate reviewing, selecting and importing clinical data for inclusion in a training data cohort for one or more target clinical inferencing models. For example, the access component 110 may access the multimodal clinical data 104 at the clinical data sources 102 via one or more wired or wireless communication networks (e.g., the Internet, an intranet, etc.) and provide for reviewing the multimodal clinical data 104 by the selection component 112 to facilitate selecting and importing the appropriate multimodal datasets for inclusion in the training data cohort. The selection component can 112 select datasets from the multimodal clinical data 104 based on the datasets respectively comprising subsets of the multimodal clinical data 104 that satisfy criteria determined to be relevant to the clinical processing task of the target clinical inferencing model 132/132′. The importing component 114 can further import the selected datasets into local memory associated with the multimodal training data cohort generation module 108 for additional processing by the selection component 112 and the cohort curation component 116 in association with further sub-selecting and refining the selected datasets into a final training data cohort that may be stored in the training data cohort database 120 and used by the training module 128 to train and/or develop the clinical inferencing model 132.

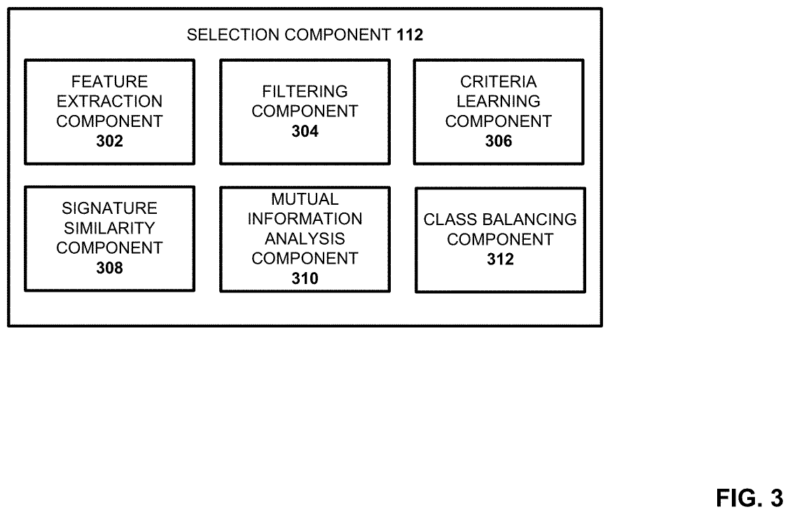

In some embodiments, at least some of the criteria used by the selection component 112 to select the datasets from the multimodal clinical data 104 may be predefined for the clinical processing task and/or the target clinical inferencing model 132/132′ in the training data cohort policy database 118. In this regard, the training data cohort policy database 118 may include training data cohort curation policy information for each (or in some implementations one or more) of the clinical inferencing models included in the clinical inferencing model database 134. The policy information can define the rules and/or policies for selecting the appropriate training data cohort datasets for each clinical inferencing model and/or for defined clinical inferencing tasks. For example, policy information may define the specific type or types of data to be included in the training data cohort datasets, and/or the specific features and/or feature values for the data. The policy information may also define one or more selection algorithms and/or models to be applied by the selection component 112 to filter and sub-select the datasets. As described in greater detail infra with reference to FIG. 3, these algorithms and/or models may include rule-based algorithms as well as machine learning algorithms/models. The selection criteria, rules and/or policies for each clinical inferencing model may vary and be tailored to the particular clinical inferencing task the respective models are adapted to perform.

In accordance with the embodiment shown in FIG. 1, the selection component 112 selects initial multimodal datasets 106 from the multimodal clinical data 104 for importing to the multimodal training data cohort generation module 108 by the importing component 114. These initial multimodal datasets 106 may be selected based on some initial selection criteria defined for the clinical inferencing model in the training data cohort database 118. For example, the initial criteria may define initial inclusion and/or exclusion criteria for the initial multimodal datasets 106. The inclusion and/or exclusion criteria may be defined by a number of parameters of choice depending on the clinical inferencing task. For example, the inclusion/exclusion criteria may define the type or types of data to be included in each initial multimodal dataset, preferred or required features of the data, and/or preferred or required feature values. Because each of the initial multimodal datasets 106 satisfy the inclusion/exclusion criteria, each or the initial multimodal datasets 106 are considered similar. For example, in some embodiments, the training data cohort policy for the model may define one or more similarity metrics for selecting the initial multimodal datasets that requires the respective datasets to reflect a similar anatomy, pathology and/or diagnosis. The similarity metrics can also require all the multimodal data within each individual initial multimodal dataset (and/or the final filtered datasets) to satisfy one or more similarity metrics based on all the multimodal data reflecting a similar anatomy, pathology and/or diagnosis. For instance, if the datasets for a patient includes imaging data that reflects a one diagnosis yet laboratory data that reflects a different diagnosis, the entire dataset can be removed from the training data cohort.

In some embodiments, the multimodal clinical data 104 comprises sets of different types of clinical data for a plurality of different patients/subjects, and the initial selection criteria can group the data for inclusion in each of the initial multimodal datasets 106 based on patient/subject similarity. For instance, in one implementation in which the clinical inferencing model 132/132′ is adapted to perform an image analysis task on medical images (e.g., a diagnosis/classification task, a segmentation task, a disease quantification task, etc.), the initial inclusion criteria can include medical image data from multiple imaging modalities captured for the same subject. With these embodiments, the initial multimodal datasets 106 can be grouped by patient, wherein all of the multimodal data included in each dataset is for the same patient, and wherein each of the initial multimodal datasets 106 comprises multimodal data for a different patient (e.g., dataset 1 comprises data for patient 1, dataset 2 comprises data for patient 2, dataset 3 comprises data for patient 3, and so on). With these embodiments, the selection component 112 can traverse the clinical data sources 102 to find relevant (e.g., as defined by the cohort selection policy for the target clinical inferencing model) clinical and non-clinical data for a same patient using one or more identifiers for the patient. The one or more patient identifiers may be anonymized and/or the cohort curation component 116 may anonymize the patient data in association with extraction of the patient data from the clinical data sources 102 for storage in the training data cohort database. Additionally, or alternatively, clinical and non-clinical information for individual patients may be pre-collated and grouped as stored in the multimodal clinical data 104.

As described above with reference to FIG. 2, a variety of different clinical and non-clinical data may be included in the multimodal clinical data 104 for each patient, and the amount of data available can vary for different patients. For example, some patients may be associated with multiple different imaging studies captured in different modalities. Each imaging study can also include several different images (scan slices in CT an MR for example) with different perspectives of an anatomical region of interest and captured with different capture parameters/protocols (e.g., contrast vs. non-contrast, different reconstruction kernel sizes, different MRI frequencies, different scanner devices, etc.). This imaging data needs to be analyzed and filtered to select the best images for training the clinical inferencing model 132. Additionally, a variety of other relevant clinical and non-clinical data (e.g., analysis and reports data 202, demography data 210, disease data 208, etc.) may be available for the patient which can provide additional input parameters for the clinical inferencing model in combination with the imaging data to improve the model performance. These additional data inputs may also be analyzed and filtered to select the optimal multimodal dataset for training the model.

In this regard, initial selection criteria can define at least some high-level filtering parameters for selecting the appropriate patient/subjects and the appropriate subset of data for the patient/subjects from the multimodal clinical data 104. For example, in one implementation in which the clinical inferencing model 132/132′ is adapted to classify a lung condition based at least in part on medical image data captured of the lungs, the initial inclusion criteria may include the following: 1. both CT and XR images captured for the same patient, 2. the CT and XR images to be captured within a 48 hour time window, 3. the images to include non-contrast images, and 4. the images to have a slice thickness of 3.0 millimeters or less. It should be appreciated that the above noted initial inclusion criteria is merely exemplary. In this regard, the initial inclusion criteria can be based on a variety of parameters regarding the type or types of clinical and non-clinical data for inclusion in the initial multimodal datasets 106, characteristics of the data, and characteristics of patients/subjects associated with the data (e.g., including demographic parameters and medical condition/disease related parameters, and so on).

In accordance with the embodiment shown, the importing component 114 can import the initial multimodal datasets 106 selected based on the initial criteria. The selection component 112 can further perform a deeper evaluation of the initial multimodal datasets to further down-select or filter the initial multimodal datasets 106 based on diverse features extracted from or otherwise associated with the data included in the initial multimodal datasets 106 and additional criteria for the training data cohort. For example, in implementations in which the initial multimodal datasets 106 include medical images, the additional criteria may be related to image quality (e.g., removing images with foreign objects, motion artifacts and/or other imaging artifacts that impact the image quality). The additional criteria may also be related to acquisition protocol requirements for the images (e.g., contrast/non-contrast acquisition, CT dose, capture position/orientation, capture perspective, MR sequence used, and so on). The additional criteria may also relate to geometric similarity between images included in the same dataset (e.g., for a same patient) and/or between all images included in all the datasets. As described in greater detail infra with reference to FIG. 3, geometric similarity of image data refers to the similarity of the relative size, shape and position of the anatomical region of interest reflected in two or more images. The additional criteria may also relate to biological signature similarity. For example, as also discussed in greater detail with reference to FIG. 3, the biological signature similarity can be based on similarity of pathology type, pathology subtype, disease type, disease stage, patient profile, demography, data source (e.g., site from which the data was generated), and the like.

The additional criteria used by the selection component 112 to further filter the initial multimodal datasets 106 into the final training data cohort datasets 126 for the target clinical inferencing model 132/132 may also be defined by the training data cohort policy for the target clinical inferencing model 132/132 in the training data cohort policy database 118. Additionally, or alternatively, the selection component 112 can employ one or more rule-based and/or machine learning techniques to further filter and refine the initial multimodal datasets into the training cohort datasets 126. Additional details regarding the training data cohort selection and filtering process are discussed infra with reference to FIG. 3.

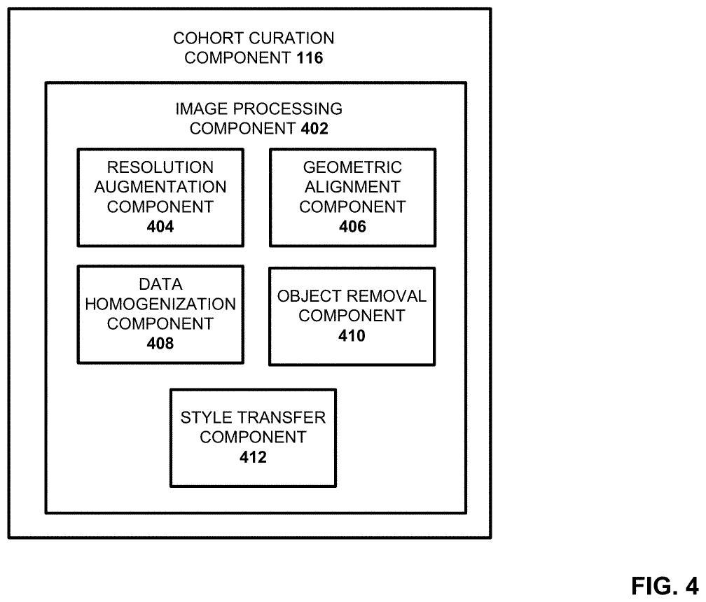

The cohort curation component 116 can generate the training data cohort for the target clinical inferencing model using the datasets selected and filtered by the selection component 112. In this regard, the training data cohort for the clinical inferencing model 132/132′ can include the training cohort datasets 126 selected by the selection component 112. For example, the cohort curation component 116 can aggregate and store the training cohort datasets 126 selected by the selection component 112 in the training data cohort database 120 with information that associated the datasets with the target clinical inferencing model 132/132. In some embodiments, the cohort curation component 116 may also pre-process the training cohort datasets 126 using one or more preprocessing tasks in association with storing the training cohort datasets 126 in the training data cohort database 120. For example, in embodiments in which the datasets include medical images, the preprocessing tasks can include (but are not limited to), image harmonization, image style transfer, image resolution augmentation, image data homogenization, and image geometric alignment. Additional details regarding these preprocessing steps are described infra with reference to FIG. 4.

As illustrated in system 100, training cohort datasets 126 generated by the multimodal training data cohort generation module 108 are employed by the training module 128 to train and/or develop the target clinical inferencing model 132/132′ to which they are tailored. For example, in the embodiment shown, the training module 128 can extract or otherwise receive the training cohort datasets 126 for the target clinical inferencing model 132 generated by the multimodal training data cohort generation module 108. The training module 128 can include a training component 130 that further trains and/or develops the clinical inferencing model 132 using the cohort multimodal datasets. The training process can vary depending on the type of the clinical inferencing model 132/132′. For example, the training component 130 can employ known supervised, semi-supervised and/or unsupervised machine learning training, testing and validation processes to train and tune the clinical inferencing model 132 until a desired performance level is achieved. The desired performance level may be based on one or more model performance evaluation metrics related to the accuracy, specificity and/or confidence of the model inference outputs.

Once at least an initial desired performance level of the clinical inferencing model 132 is achieved by the training component 130, the trained version of the clinical inferencing model can be stored in the clinical inferencing model databases 134. The inferencing module 138 can further apply the trained version of the clinical inferencing model 132′ to new datasets in the field to generate one or more inference outputs 142 for corresponding use cases and applications. In the embodiment shown, these new datasets are represented as new multimodal datasets 136. The new multimodal datasets 136 may be selected/retrieved from the clinical data sources 102 using the same or similar criteria applied by the multimodal training data cohort generation module 136 to generate the training cohort datasets 126. For example, the inferencing module 138 can include a model application component 140 that applies the clinical inferencing model 132′ to the selected multimodal datasets 136 to generate the inference outputs 142. The model application component 140 and/the inferencing module 138 can further employ the data selection functions provided by the multimodal training data cohort generation module 108 to select/extract the appropriate input data sets (e.g., the new multimodal datasets 136) for input into the clinical inferencing model 132′. In this regard, the inferencing module 138 can employ the access component 110, the selection component 112, and/or the importing component 114 (and/or instances thereof), along with the training data cohort selection policy defined for the clinical inferencing model 132′ in the training data cohort policy database 118 to select, filter and preprocess the new multimodal datasets for input into the clinical inferencing model 132′. The model application component 140 and/the inferencing module 138 can also employ the data preprocessing functions (e.g., image resolution augmentation, image geometric alignment, data homogenization, etc.) provided by cohort curation component 116 to preprocess the selected multimodal datasets 136 as needed prior to into the clinical inferencing model 132′.

The multimodal training data cohort generation module 108 can further include cohort optimization component 122 to perform a continuous learning process based on evaluation of the inference outputs 142 to learn how to refine/tailor the training data cohort for the target clinical inferencing model 132/132′ to improve the model performance using cohort optimization component 122. To facilitate this end, the cohort optimization component 122 can include a learning component 124 that evaluates performance of the clinical inferencing model 132 on the multimodal new datasets to learn one or more features of the new datasets associated with a measure of poor model performance. For example, the learning component 124 can identify those new multimodal datasets 136 that are close to and/or far from the model decision boundary (and thus attributed inaccurate and/or unreliable inference results). For instance, in some embodiments, the clinical inferencing model 132′ can be configured to generate a confidence score for each inference output 142 that reflect a degree of confidence in the accuracy of the inference output 142. With these embodiments, the learning component 124 can identify boundary datasets associated with low confidence scores (e.g., relative to a defined threshold). Additionally, or alternatively, the learning component 124 can receive feedback regarding the accuracy of the inference outputs 142. For example, the accuracy feedback may be manually provided (e.g., based on manual review of the inference outputs) and/or determined automatically based on retrospective analysis of new patient data received for the patient that confirms or negates the accuracy of the model inference output. With these embodiments, the learning component 124 can identify boundary datasets included amongst the new multimodal datasets 136 that are associated with inaccurate inference outputs 142 (e.g., relative to a defined measure of accuracy). The learning component 124 can further evaluate the identified boundary datasets in view of the non-boundary datasets and the current training data cohort selection policy for the clinical inferencing model 132 to determine distinguishing features/attributes of the boundary datasets. The learning component 124 can employ various machine learning techniques to determine/learn the distinguishing features (e.g., k-means, clustering, regression analysis, random forest, decision trees, etc.).

The cohort optimization component 122 can further refine the training data cohort selection policy/criteria for the target clinical inferencing model 132 as defined in the training data cohort policy database 118 to pull additional input training datasets from the multimodal clinical data 104 for inclusion in the training data cohort for the model that correspond to the boundary cases. The training module 128 can further retrain and tailor the clinical inferencing model 132 using the refined training data cohort to generate an updated version of the clinical inferencing model 132. This continual learning process can be regularly and/or continuously performed after initial model training and development to generate updated versions of the clinical inferencing model that provide greater accuracy and/or specificity. In this regard, the process for defining and refining the training data cohort selection policy for a particular clinical inferencing model can involve a continuous learning process that is automatically performed by the leaning component 124 and the cohort optimization component 122. For example, in some embodiments, a baseline or default cohort selection policy can be defined for the target clinical inferencing model prior to initial training and development. This baseline/default cohort selection policy can be used by the multimodal training data cohort generation module 108 to generate a first training data cohort for the model which is then used by the training module 128 to train and develop the target clinical inferencing model to perform the clinical inferencing task. Once a default level of model performance is achieved by the training component 130, the inferencing module 138 can apply the model to the new multimodal datasets selected using the baseline/default cohort selection policy. The cohort optimization component 122 can then refine the baseline/default cohort selection policy using the techniques described above and the multimodal training data cohort generation module 108 can select a new batch of multimodal training datasets according to the updated cohort selection policy criteria. This new batch of multimodal training datasets can then be used by the training module 128 to retrain and fine tune the clinical inferencing model 132 until a higher level (e.g., relative to the default level) of model performance is achieved. This process can be repeated continuously over time until model convergence is achieved.

FIG. 3 presents an example selection component 112 for selecting clinical ML model input data sets in accordance with one or more embodiments of the disclosed subject matter. With reference to FIGS. 1 and 3, as noted above, the selection component 112 can provide for selecting datasets (e.g., the initial multimodal datasets 106 and the (final) training cohort datasets 126) from the multimodal clinical data 104 based on the datasets respectively comprising subsets of the multimodal clinical data that satisfy criteria determined to be relevant to the clinical processing task of the target clinical inferencing model 132/132′. To facilitate this end, the selection component 112 can include feature extraction component 302, filtering component 304, criteria learning component 306, signature similarity component 308, mutual information analysis component 310 and class balancing component 312.

The feature extraction component 302 can extract diverse features from the multimodal clinical data that can be used by the selection component 112 to identify the subsets. For example, in some embodiments, the training data cohort selection policy for the clinical inferencing task/model as provided in the training data cohort policy database 118 can define initial selection criteria for the initial multimodal datasets 106 that identifies one or more required features for the initial multimodal datasets 106. For example, the initial selection criteria may define the type or types of data to be included in each of the initial multimodal datasets 106 and/or required features of the type or types of data. With these embodiments, the feature extraction component 302 can extract these one or more features from the multimodal clinical data 104 as stored at the one or more clinical data sources 102 to identify the initial multimodal datasets 106 and the importing component 114 can import the initial multimodal datasets 106 from the one or more clinical data sources 102 based on the initial multimodal dataset comprising the one or more features.

Additionally, or alternatively, the feature extraction component 106 can process the initial multimodal datasets 106 once imported into local memory of the multimodal training data cohort generation module 108 to extract a variety of additional features from the data included in the initial multimodal datasets 106 that can be used to further filter (e.g., sub-select) and refine the initial multimodal datasets 106 into the training cohort datasets 126. The feature extraction component 106 can employ a variety of text-based and image-based feature detection/extraction algorithms to identify and extract features from image and/or text data included in the initial multimodal datasets 106. For example, the feature extraction component 302 can employ various natural language processing (NLP) to identify and extract key terms and values from text data included in the multimodal datasets 106, such as text data included in clinical reports (e.g., laboratory reports, radiologist reports, clinical notes, etc.), EMRs, metadata associated with medical imaging studies and the like. The feature extraction component 302 can also employ image-processing feature detection algorithms to extract relevant features from medical images included in the initial multimodal datasets 106. Some example, suitable feature extraction techniques that can be employed by the feature extraction component 106 include but are not limited to: local binary pattern, gray level co-occurrence matrix, gray level run length method, Harlik features, Gabor texture features, learning vector quantization, symbolic dynamic filtering, principal component analysis, and independent component analysis.

The filtering component 304 can filter the initial multimodal datasets 106 based on the extracted features associated therewith and additional (second) selection criteria for the training data cohort datasets 126 that defines or indicates required or preferred features/feature values for the training data cohort datasets 126. As described above, in some embodiments, this additional selection criteria may be predefined and included in the policy for the clinical inferencing task/clinical inferencing model 132/132′. Additionally, or alternatively, the criteria learning component 306 can employ principles of artificial intelligence and machine learning to learn the additional criteria based on analysis and comparison of the features of the data included within a same dataset of the initial datasets and analysis and comparison of the initial datasets to one another. The patient flow criteria learning component 306 can perform learning associated with the initial multimodal datasets 106 explicitly or implicitly.

Learning and/or determining inferences by the learning component 306 can facilitate identification and/or classification of different patterns associated with the initial multimodal datasets 106, determining one or more rules associated with filtering the initial multimodal datasets 106, and/or determining one or more relationships among multimodal data included within the initial multimodal datasets 106. The criteria learning component 306 can also employ an automatic classification system and/or an automatic classification process to facilitate identification and/or classification of different patterns associated with the initial multimodal datasets 106, determining one or more rules associated with filtering the initial multimodal datasets 106, and/or determining one or more relationships among multimodal data included within the initial multimodal datasets 106. For example, the criteria learning component 306 can employ a probabilistic and/or statistical-based analysis (e.g., factoring into the analysis utilities and costs) to learn one or more patterns associated with the initial multimodal datasets 106, determining one or more rules associated with filtering the initial multimodal datasets 106, and/or determining one or more relationships among multimodal data included within the initial multimodal datasets 106. The criteria learning component 306 can employ, for example, a support vector machine (SVM) classifier to facilitate learning patterns associated with the initial multimodal datasets 106, determining one or more rules associated with filtering the initial multimodal datasets 106, and/or determining one or more relationships among multimodal data included within the initial multimodal datasets 106. Additionally or alternatively, the criteria learning component 306 an employ other classification techniques associated with Bayesian networks, decision trees and/or probabilistic classification models. Classifiers employed by the criteria learning component 306 can be explicitly trained (e.g., via a generic training data) as well as implicitly trained (e.g., via observing user behavior, receiving extrinsic information). For example, with respect to SVM's that are well understood, SVM's are configured via a learning or training phase within a classifier constructor and feature selection module. A classifier is a function that maps an input attribute vector, x=(x1, x2, x3, x4, xn), to a confidence that the input belongs to a class—that is, f(x)=confidence(class).

In an aspect, the criteria learning component can include an inference component that can further enhance automated aspects of the criteria learning component 306 utilizing in part inference-based schemes to facilitate learning one or more patterns associated with the initial multimodal datasets 106, determining one or more rules associated with filtering the initial multimodal datasets 106, and/or determining one or more relationships among multimodal data included within the initial multimodal datasets 106. The criteria learning component 306 can employ any suitable machine-learning based techniques, statistical-based techniques and/or probabilistic-based techniques. The criteria learning component 306 can additionally or alternatively employ a reduced set of factors (e.g., an optimized set of factors) to facilitate providing a most accurate machine learning model for predicting census in respective medical inpatient units. For example, the patient flow component 104 can employ expert systems, fuzzy logic, SVMs, Hidden Markov Models (HMMs), greedy search algorithms, rule-based systems, Bayesian models (e.g., Bayesian networks), neural networks, other non-linear training techniques, data fusion, utility-based analytical systems, systems employing Bayesian models, etc. In another aspect, the criteria learning component 306 can perform a set of machine learning computations associated with the initial multimodal datasets 106. For example, the criteria learning component 306 can perform a set of clustering machine learning computations, a set of decision tree machine learning computations, a set of instance-based machine learning computations, a set of regression machine learning computations, a set of regularization machine learning computations, a set of rule learning machine learning computations, a set of Bayesian machine learning computations, a set of deep Boltzmann machine computations, a set of deep belief network computations, a set of convolution neural network computations, a set of stacked auto-encoder computations and/or a set of different machine learning computations. The learned selection/filtering criteria for filtering the initial multimodal datasets 106 into the training cohort datasets 126 can be stored in the selection policy (e.g., in the training data cohort selection policy database 118) for the target clinical inferencing model 132/132 and/or the clinical inferencing task of the model.

In certain embodiments, the one or more patterns associated with the data included in each initial multimodal datasets 106 can be configured as one or more digital fingerprints (e.g., one or more digital signatures) that represents one or more digital patterns associated with data. A digital fingerprint can be a string of bits associated with a portion of the respective initial multimodal datasets 106. In certain implementations, a digital fingerprint can comprise a sequence of sub-fingerprints associated with different patterns in data associated with the respective initial multimodal datasets 106. Furthermore, a digital fingerprint can uniquely identify and/or convey a pattern in the data associated with the respective initial multimodal datasets 106. For example, a digital fingerprint can be a data element that encodes a pattern in the data associated with the respective initial multimodal datasets 106.

The signature similarity component 308 can employ one or more digital fingerprinting techniques (e.g., one or more digital fingerprint algorithms) to map at least a portion of the data associated with the respective initial multimodal datasets 106 into one or more digital fingerprints. For example, the signature similarity component 308 can employ a hash technique to generate one or more digital fingerprints associated the data included in each of the initial multimodal datasets 106. In another example, the signature similarity component 308 can employ a locality sensitive hashing technique to generate the one or more digital fingerprints. In yet example, the signature similarity component 308 can employ a random hashing technique to generate the one or more digital fingerprints. In an implementation, a digital fingerprint can comprise min-hash values associated with a portion of the patient flow data 116. For example, a digital fingerprint can comprise a vector of min-hash values associated with a portion of the patient flow data 116. In another example, a digital fingerprint can comprise a band of min-hash values associated with a portion of initial multimodal datasets 106. In yet another example, a digital fingerprint can comprise a locality-sensitive hashing band of min-hash values associated with a portion of the initial multimodal datasets 106. However, it is to be appreciated that other types of digital fingerprinting techniques and/or hashing techniques can be employed to generate a digital fingerprint associated with the respective initial multimodal datasets 106.

In some embodiments in which each of the initial multimodal datasets 106 respectively include one or more medical image of a same anatomical ROI the signature similarity component 308 can be configured to generate geometric signatures for each of the medical images. A geometric signature for a medical image can correspond to a digital fingerprint that represent one or more geometric patterns in the medical images. For example, the geometric signature for a medical image can be based on the relative size, shape and position of one or more anatomical features of reference in the ROI, such as the relative shape, size and/or position of an organ of interest. The signature similarity component 308 can further compare the geometric signatures associated with each image to one another and/or a reference geometric signature to determine a measure of geometric similarity between the images and/or the reference image. For example, geometric signatures may be determined for each image in the dataset and the measure of geometric similarity can be calculated based on a degree of variation between the geometric signatures between the images. Additionally, or alternatively, the geometric similarity component 308 can determine a measure of geometric similarity between images through transforms like non-rigid registration (NRR) or other methods such as FOV matching on ROI for given use case. The filtering component 304 can further employ the measure of geometric similarity to filter the initial datasets to remove images and/or entire datasets based on the images being too geometrically dissimilar.

In particular, medical image processing models adapted to perform clinical inferencing tasks on medical images (e.g., diagnosis tasks, anomaly detection tasks, segmentation tasks, risk/staging tasks, etc.) can be very sensitive to appearance variations in the input medical images. These appearance variations can be caused by a variety of imaging factors (e.g., image capture protocol, dose usage, exposure setting, photon receiving materials, FOV), demography, contrast vs. non-contrast, operating technician skill, etc.). Thus, in some embodiments, the training data cohort selection policy for the target clinical inferencing model 132/132′ can include a geometric similarity requirement for the images to be included in the training cohort datasets 126 and the filtering component 304 can be configured to filter out images and/or entire datasets with images that fail to satisfy the geometric similarity criteria.

In some embodiments the geometric similarity criteria can be based on the degree of geometric similarity between two or more input images included in each dataset. For example, the training data cohort policy can require each final training data cohort dataset to include N number of input images that are geometrically similar, wherein N can include an integer greater than one. For instance, the policy may require a first image from a first capture modality (e.g., XR) and a second image from a second capture modality (e.g., CT) of the same anatomical region of a patient captured within a defined time window of one another (e.g., 48 hours) that are geometrically similar. In accordance with this example, the signature similarity component 308 can determine geometric signatures for each of the images included in the initial datasets. The signature similarity component 308 can further compare the geometric signatures of the XR images with that of the CT images to determine measures of geometric similarity between pairs of XR and CT images. Assuming an initial dataset includes a plurality of image pairs, in some implementations the filtering component 308 can filter the images included in the dataset to select one image pair that is the most geometrically similar (e.g., whose geometric signatures are the most similar). The filtering component 304 can also further remove entire datasets from the initial multimodal datasets 106 that fail to include at least one image pair whose geometric signatures are sufficiently similar to satisfy the geometric similarity requirement.

Additionally, or alternatively, the geometric similarity criteria can be based on the degree of geometric similarity between images included in separate datasets. For example, the geometric similarity criteria for the policy can require all of the training cohort datasets 126 to include images that have geometric signatures that are geometrically similar (e.g., relative to a defined degree of similarity) to one another and/or a reference image geometric signature. With these embodiments, the filtering component 304 can remove datasets from the initial datasets 106 that fail to include a medical image or medical images that have at least a defined degree of geometrical similarity to one another and/or the reference image.

The signature similarity component 308 may also be configured to determine and evaluate biological signatures for the initial datasets 106 and the filtering component 304 can be configured to select datasets from the initial datasets 106 for inclusion in the final training data cohort based on the datasets satisfying a biological similarity criteria. In this regard, a biological signature for a dataset can provide a feature vector representation of one or more relevant biological and/or physiological features associated each of the initial datasets. For example, the biological signature for an initial dataset may provide a reduced dimensional representation of the pathology, pathology subtype, disease, disease stage, clinical patient condition, physiological parameters of the patient, and so on represented by the clinical data included in the dataset. In some implementations, the biological signature can also represent non-clinical features extracted from the patient profile/demography data for the patient represented by the data in the dataset. With these embodiments, the filtering component 304 can be configured to remove datasets from the initial datasets 106 whose biological signature is significantly dissimilar from other datasets (e.g., relative to a defined degree of deviation) and/or significantly dissimilar to a reference biological signature (e.g., relative to a defined degree of deviation).

The mutual information analysis component 310 can further evaluate mutual information included in and/or associated with each of the initial datasets 106 to sub-select only those datasets whose mutual information reflects a consistent anatomy, pathology or diagnosis. For example, the mutual information may be based on data included in the initial multimodal datasets 106 and/or the training data cohort datasets 126 that will be used as input to the clinical inferencing model 132/132′. The mutual information may also be based on auxiliary data included in the initial multimodal datasets 106 and/or the training cohort datasets 126 that will not be used as input to the clinical inferencing model 132/132′. For example, this auxiliary data may be included in the datasets and used by the selection component 112 to filter and evaluate the datasets only. Additionally, or alternatively, this auxiliary data may not be imported from the clinical data sources 102. For example, the selection component 112 can access and evaluate this auxiliary data as located in the one or more clinical data sources 102 to facilitate filtering the datasets without extracting/importing the auxiliary data.

In this regard, the mutual information analysis component 310 can identify inconsistencies between clinical information included in and/or associated with individual datasets of the initial multimodal datasets (e.g., as provided in the one or more clinical data sources) and remove those datasets that fail to satisfy one or more similarity metrics. For example, the one or more similarity metrics may be based on the datasets reflecting a consistent diagnosis, anatomy, staging, pathology, or other similar features. With these embodiments, the inconsistencies may be determined based on inconsistencies in diagnosis, anatomy, staging, pathology, and/or other information that is relevant to the staging, anatomy, diagnosis, pathology, etc. For instance, assume the initial datasets include medical images from different studies performed with different capture modalities. The mutual information analysis component 310 can evaluate mutual information provided in the radiologist reports for each of the studies to determine whether the mutual information indicates a same or similar diagnosis between the studies and remove the datasets if not. The mutual information can also include laboratory data, physiological parameter data, and other clinical report data and findings associated with the imaging studies. For example, assume the clinical inferencing model 132/132′ is being trained to diagnose lung disease attributed to COVID-19 based on one or more chest XR images of the patient and thus the initial datasets include chest XR images of patients with and without the lung disease. Mutual information associated with these initial datasets may include diagnosis and staging data provided in the radiologist reports, laboratory testing results, and measure physiological data for the patient. In one example, if the chest XR image report indicates a clean diagnosis (e.g., no COVID-19 lung disease present), but the laboratory data indicates the patient tested positive for COVID-19, then the mutual information analysis component 310 can remove the dataset from the training data cohort based on this inconsistent diagnosis across the multimodal data, unless the clean XR diagnosis is supported by other mutual data modalities, like SpO₂ or CT or some other forms of information, which hints at lung involvement or expert radiologist assessment.

The class balancing component 312 can further perform a class balancing analysis in association with filtering the initial multimodal datasets 106 to ensure the multimodal training data cohort has a sufficient distribution of the different classes to be evaluated. For example, the assume the clinical inferencing model 132/132 is being trained to classify presence and absence of COVID-19 chest disease in XR images. A balanced training data cohort should include a balanced ratio of both clean and positive XR cases. The training dataset classes will vary depending on the type of clinical inferencing task at hand. For example, the different classes may be based on pathology, pathology sub-type, pathology staging, diagnosis, lesion type, lesion size, lesion location and son on. In this regard, the class balancing component 312 can remove those initial datasets 106 for overrepresented classes to balance the distribution of the datasets included in the final training data cohort for the model.

FIG. 4 presents an example cohort curation component 116 in accordance with one or more embodiments of the disclosed subject matter. The cohort curation component 116 can perform various pre-processing steps on the final down selected training cohort datasets 126 to prepare them for processing by the clinical inferencing model 132/132′. In particular, in implementation in which the training data cohort datasets include on or more medical images, the cohort curation component 116 can include image processing component 402 to perform various image processing functions to augment the images to compensate for differences in resolution, geometric alignment, scanner and other similar features. To facilitate this end, the image processing component 402 can include resolution augmentation component 404, geometric alignment component 406, data homogenization component 408, object removal component 410 and style translation component 412.

The resolution augmentation component 404 can perform an image resolution augmentation process to generate image data with similar resolutions (e.g., spatial and temporal, wherever applicable), in cases of gross mismatch (e.g., relative to a defined deviation). The resolution augmentation component 404 can employ various existing resolution enhancement techniques to transform the image data into images with a same or similar resolution. Some suitable techniques may include image processing methods, such as image sharpening by a high-pass filter, image deblurring by a Laplacian filter and the Richardson—Lucy algorithm, and image deblurring using one or more deep learning-based enhancement models trained to transform low/normal resolution input images into higher resolution output images.

The geometric alignment component 406 can perform an image data alignment/registration process to align image data such that anatomical landmarks and/or ROIs for the given tasks are geometrically similar. The image registration process can involve shifting or morphing the geometry (e.g., shape, size, orientation, FOV, etc.) of an image to be more similar to that of one or more reference images such that all the images in the datasets have a same or similar geometrical alignment.

The data homogenization component 408 can perform a data homogenization processes such that the data acquired from different scanners and acquisition protocols are visually similar. For example, the data homogenization component 408 can perform one or more image harmonization techniques to harmonize the image data included in a same dataset and/or to harmonize all the images include in the training data cohort (e.g., energy band harmonization, sub-band image harmonization, etc.). For instance, the data homogenization component 408 can harmonize images included in the training data cohort datasets with one or more reference images to make all the images look more similar in appearance to the one or more reference images. In this regard, the image harmonization process can involve adapting/adjusting the visual appearance of the images to be more similar to that of the one or more reference images, resulting in transformation of the images into harmonized images.

The object removal component 410 can perform an object removal process to remove foreign objects, motion artifacts and/or other unwanted artifacts from the medical images that impact the image quality. For example, the object removal process can involve removing non-body parts included in the 3D image data (e.g., the imaging table or the like), removing anatomical features/parts outside the region of interest, and the like.

The style transfer component 412 can change the appearance of images included in the datasets image to appear more similar to one or more reference images. For example, in some embodiments, the style transfer component 412 can employ a style translation model configured to translate or transform an image captured using one modality into the appearance style of a reference image captured with a different capture modality such that all the images within the datasets have a similar style appearance. This style translation model can include a previously trained machine learning model, such as a deep learning model, an autoencoder model, a generative adversarial network (GAN) model, a convolutional neural network (CNN) model, or another type of machine learning model.

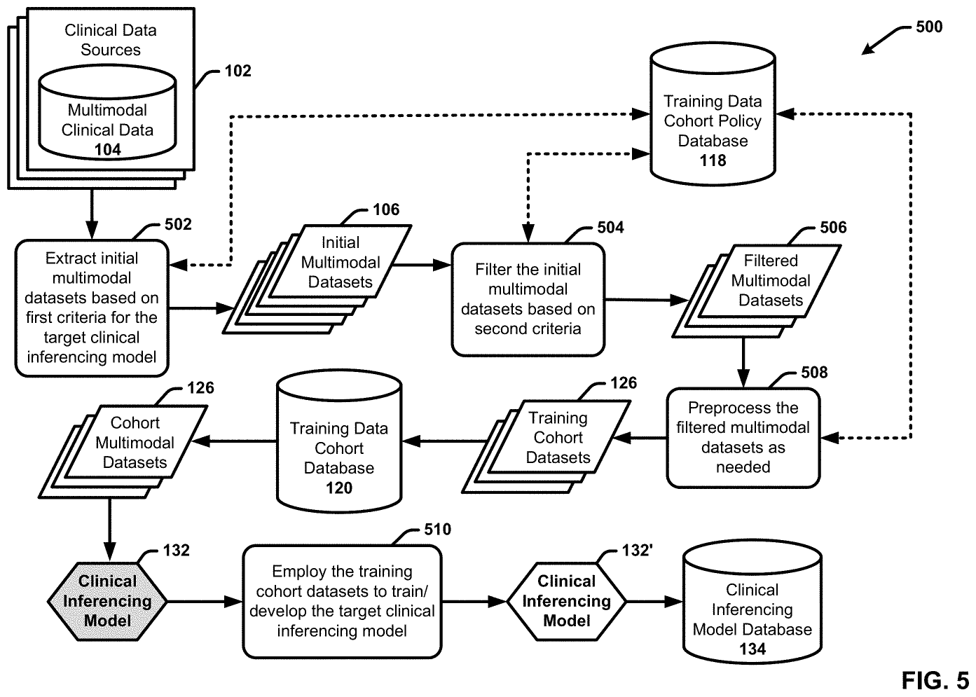

FIG. 5 presents a high-level flow diagram of an example process 500 for generating multimodal training data cohorts tailored to specific clinical ML model inferencing tasks in accordance with one or more embodiments of the disclosed subject matter. In this regard, process 500 demonstrates an example method that may be performed by the multimodal training data cohort generation module 108 using the access component 110, the selection component 112, the importing component 114 and the cohort curation component 116. Repetitive description of like elements employed in respective embodiments is omitted for sake of brevity. In the embodiment shown, the solid arrows indicate directional flow of process 500 and the dashed arrow lines indicate communicative data connections between the respective data structures and the corresponding processing steps.

With reference to FIGS. 1 and 5, in accordance with method 500, at 502, initial multimodal datasets 106 are extracted from the pool of multimodal clinical data 104 based on first criteria (e.g., via the importing component 114 and the selection component 112). The first filter criteria may include one or more predefined criteria for the data to be included in the initial multimodal datasets 106 as predefined in a training data cohort policy for the target clinical inferencing model and/or the clinical inferencing task of the model as provided in the training data cohort policy database 118. For instance, in accordance with one example use case in which the clinical inferencing task involves classification of a lung disease based at least in part on analysis of medical image data captured of the lungs, the first filter criteria may require each of the initial datasets 106 to include chest medical imaging studies captured in both CT and XR withing a 48-hour time window for patients with and without the lung disease. The first filter criteria may also define some additional inclusion/exclusion criteria for the image data, such as criteria related to required or preferred acquisition parameters/protocols used for the imaging studies, required or preferred demographic profiles of the patients, and so on.

At 504, the initial multimodal datasets are further filtered based on second criteria, which may also be defined in policy for the target clinical inferencing model 132/132′ as included in the training data cohort policy database 118. This can involve removing one or more datasets from the initial datasets that fail to satisfy the second criteria, resulting in filtered multimodal datasets 506. For example, the second criteria may include predefined criteria related to quality of the images, annotations associated with the images, and other features associated with the images and/or the patients/subjects associated with the images. In this regard, at 504, the selection component 112 may extract and evaluate additional features associated with the images and/or the patients/subjects associated with the images to further filter and sub-select the initial datasets (e.g., using feature extraction component 302, filtering component 304, signature similarity component 308, mutual information analysis component 310 and class balancing component 312). For example, in some implementations, this can involve performing imaging processing tasks to identify and remove images that fail to satisfy one or more image quality criteria, geometric alignment criteria, and/or other criteria for the images. This can also involve determining and evaluating biological signatures associated with the images/patients and removing those datasets that fail to satisfy defined biological signature similarity criteria. This can also involve employing mutual information associated with the patients and the images to further filter the initial datasets. This can also involve performing class balancing via the class balancing component 312 to remove datasets in over-represented classes.

At 508, the system may preprocess the filtered multimodal datasets 506 as needed (e.g., using cohort curation component 116). Pre-processing requirements for the filtered multimodal datasets 506 may also be defined in the training data curation policy for the clinical inferencing model 132/132′ as provide in the training data cohort policy database 118. For instance, in furtherance to the example use case described above in which the datasets comprise medial images, the system may perform image processing tasks such as resolution augmentation, geometric alignment, and data homogenization. Additionally, or alternatively, some of these image preprocessing steps may be performed at 504 in order to facilitate evaluating and filtering the initial multimodal datasets 106 into the filtered multimodal datasets 506 (e.g., to determine identify and remove artifacts, to determine and evaluate geometric similarity, and the like.

The final filtered and pre-processed datasets are the training cohort datasets 126 that tailored for training the clinical inferencing model 132. These training cohort datasets 126 may be stored in the training data cohort database 120. In accordance with process 500, at 510 the training component 130 may employ the training cohort datasets 126 to train and/or develop the clinical inferencing models 132. Once trained to achieve a desired level of performance, the trained clinical inferencing model 132′ may be stored in the clinical inferencing model 134.

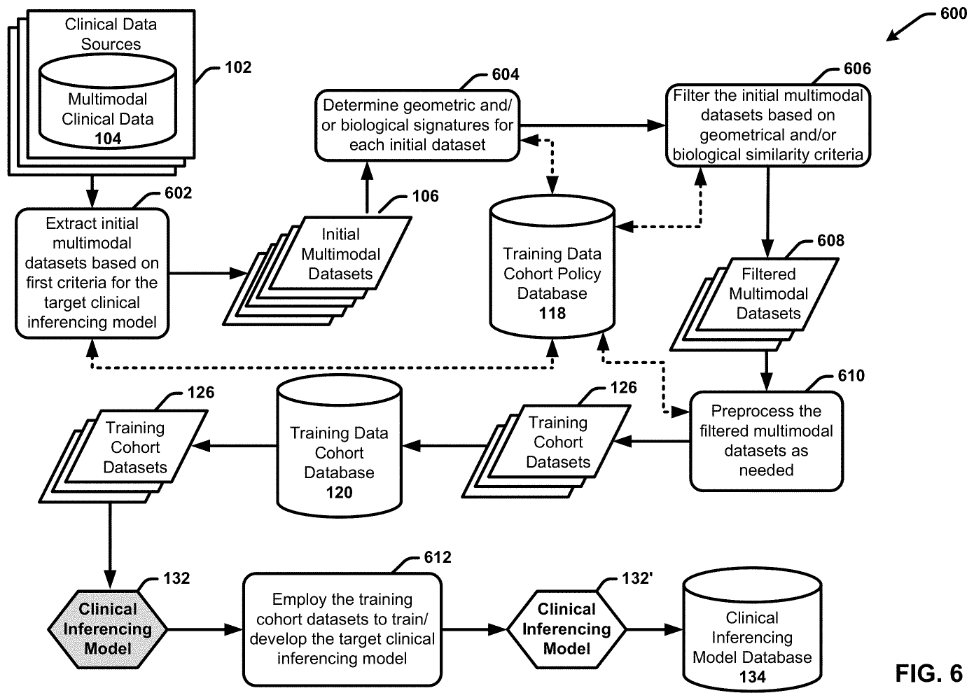

FIG. 6 presents a high-level flow diagram of another example process 600 for generating multimodal training data cohorts tailored to specific clinical ML model inferencing tasks in accordance with one or more embodiments of the disclosed subject matter. In this regard, process 600 demonstrates another example method that may be performed by the multimodal training data cohort generation module 108 using the access component 110, the selection component 112, the importing component 114 and the cohort curation component 116. Repetitive description of like elements employed in respective embodiments is omitted for sake of brevity. In the embodiment shown, the solid arrows indicate directional flow of process 600 and the dashed arrow lines indicate communicative data connections between the respective data structures and the corresponding processing steps.

With reference to FIGS. 1, 3 and 6, in accordance with method 600, at 602, initial multimodal datasets 106 are extracted from the pool of multimodal clinical data 104 based on first criteria (e.g., via the importing component 114 and the selection component 112) using same or similar techniques as described with reference to method 500. At 604, geometric and/or biological signatures are determined for each initial dataset (e.g., using signature similarity component 308) using the techniques described with reference to FIG. 3. At 606, the initial multimodal datasets are filtered into filtered multimodal datasets 608 based on geometrical and/or biological similarity criteria 606 (e.g., via the filtering component 304 and/or the signature similarity component 208 as described with reference to FIG. 3). For example, this can involve filtering images included individual datasets of the initial multimodal datasets 106 to remove images that fail to satisfy the geometric similarity criteria and/or to select a subset of the images (e.g., XR-CT image pairs) that are the most geometrically similar. This can also involve filtering out entire datasets to remove datasets with image data that fail to satisfy the geometric similarity criteria as well and/or datasets that fail to satisfy biological similarity criteria based on the biological signatures determined for the datasets. Information defining the geometric similarity criteria and/or the biological similarity criteria may be defined int the training data cohort policy for the model in the training data cohort policy database 118.

At 610, the system may preprocess the filtered multimodal datasets 608 as needed (e.g., using cohort curation component 116). Pre-processing requirements for the filtered multimodal datasets 608 may also be defined in the training data curation policy for the clinical inferencing model 132/132′ as provided in the training data cohort policy database 118. Additionally, or alternatively, some of these image preprocessing steps may be performed at 604 in order to facilitate determining the geometric and/or biological signatures. The final filtered and pre-processed datasets are the training cohort datasets 126 that tailored for training the clinical inferencing model 132. These training cohort datasets 126 may be stored in the training data cohort database 120. In accordance with process 600, at 612 the training component 130 may employ the training cohort datasets 126 to training and/or develop the clinical inferencing models 132. Once trained to achieve a desired level of performance, the trained clinical inferencing model 132′ may be stored in the clinical inferencing model 134.

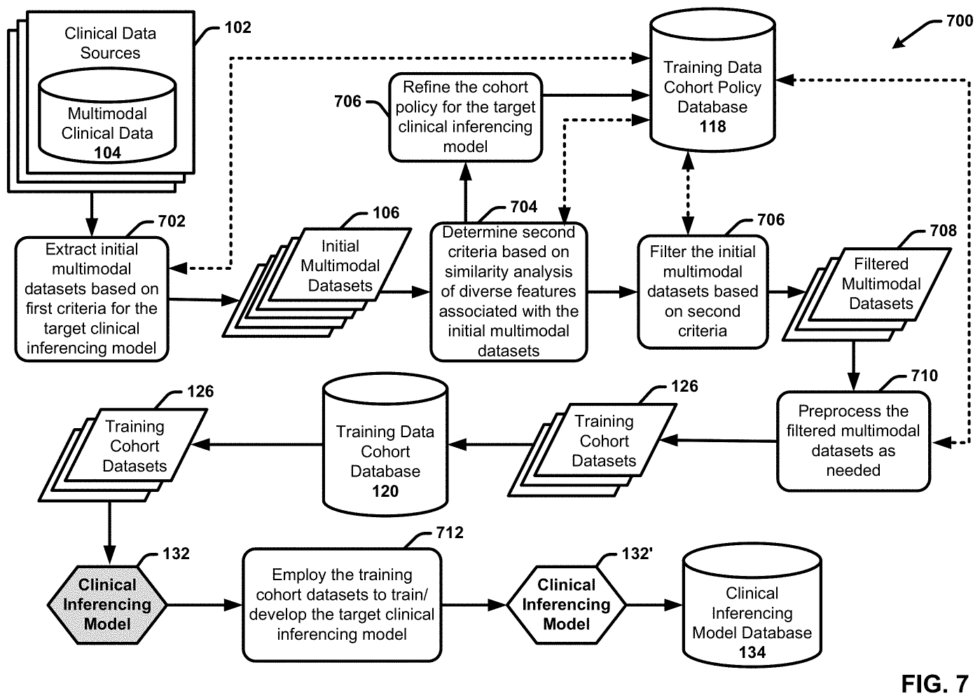

FIG. 7 presents a high-level flow diagram of another example process 700 for generating multimodal training data cohorts tailored to specific clinical ML model inferencing tasks in accordance with one or more embodiments of the disclosed subject matter. In this regard, process 700 demonstrates another example method that may be performed by the multimodal training data cohort generation module 108 using the access component 110, the selection component 112, the importing component 114 and the cohort curation component 116. Repetitive description of like elements employed in respective embodiments is omitted for sake of brevity. In the embodiment shown, the solid arrows indicate directional flow of process 700 and the dashed arrow lines indicate communicative data connections between the respective data structures and the corresponding processing steps.

With reference to FIGS. 1, 3 and 7, in accordance with method 700, at 702, initial multimodal datasets 706 are extracted from the pool of multimodal clinical data 104 based on first criteria (e.g., via the importing component 114 and the selection component 112) using same or similar techniques as described with reference to methods 500 and 600. For example, the first criteria can be pre-defined in the training data cohort policy for the clinical inferencing model 132 as included in the training data cohort policy database 118. At 704, the criteria learning component 306 can determine second filtering criteria for the clinical inferencing models 132 based on similarity analysis of diverse features associated with the initial multimodal datasets 106, as described with reference to FIG. 3. At 706, the system can refine the cohort policy for the target clinical inferencing model to include the learned second criteria (e.g., via the criteria learning component 306). At 706, the initial multimodal datasets 106 are filtered into filtered multimodal datasets 708 based on the second criteria (e.g., via the filtering component 304).

At 710, the system may preprocess the filtered multimodal datasets 708 as needed (e.g., using cohort curation component 116). Pre-processing requirements for the filtered multimodal datasets 708 may also be defined in the training data curation policy for the clinical inferencing model 132/132′ as provided in the training data cohort policy database 118. Additionally, or alternatively, some of these image preprocessing steps may be performed at 704 in order to facilitate determining the second criteria. The final filtered and pre-processed datasets are the training cohort datasets 126 that tailored for training the clinical inferencing model 132. These training cohort datasets 126 may be stored in the training data cohort database 120. In accordance with process 700, at 712 the training component 130 may employ the training cohort datasets 126 to training and/or develop the clinical inferencing models 132. Once trained to achieve a desired level of performance, the trained clinical inferencing model 132′ may be stored in the clinical inferencing model 134.

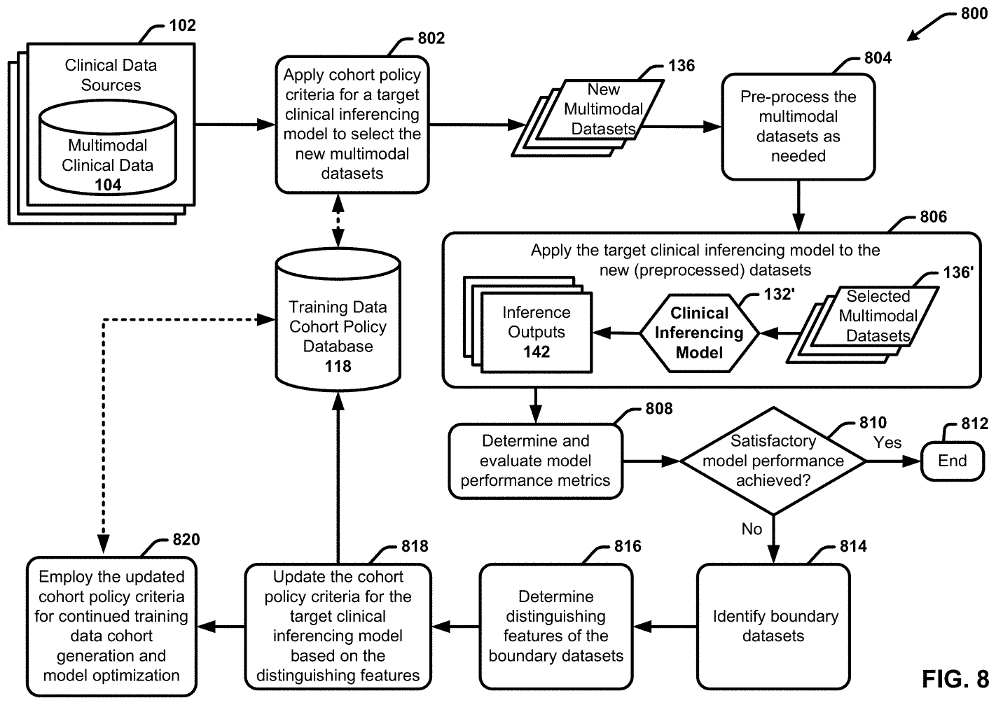

FIG. 8 presents a high-level flow diagram of an example process 800 for refining multimodal training data cohort selection for a specific clinical ML model in accordance with one or more embodiments of the disclosed subject matter. In this regard, process 800 demonstrates an example method that may be performed by the inferencing module 138 and the multimodal training data cohort generation module 108. Repetitive description of like elements employed in respective embodiments is omitted for sake of brevity. In the embodiment shown, the solid arrows indicate directional flow of process 800 and the dashed arrow lines indicate communicative data connections between the respective data structures and the corresponding processing steps.

At 802, the cohort policy criteria (e.g., as provided in the training data cohort policy database 118) for a target clinical inferencing model can be applied to select the new multimodal datasets 136 for processing by the trained clinical inferencing model 132′ (e.g., via the selection component 112). At 804, the new multimodal datasets can be pre-processed as needed (e.g., via the image processing component 402 to perform resolution augmentations, geometric alignment, data homogenization, object removal, and so on). At 806, the modal application component 140 can apply the target clinical inferencing model 132′ to the new (optionally pre-processed) datasets 136 to generate corresponding inference outputs 142. At 808, the learning component 124 can determine and evaluate model performance metrics. For example, the learning component 124 can determine and/or receive feedback regarding the accuracy of the inference outputs 142 and/or the model confidence in the inference outputs 142. At 810, the learning component can determine whether satisfactory model performance has been achieved based on the model performance metrics. If so, then process 800 can end at 812 (e.g., model convergence achieved). If not, then at 814, the learning component 124 can identify the boundary datasets. At 816, the learning component can determine distinguishing features of the boundary datasets. At 818, the cohort optimization component 124 can update the training data cohort selection policy criteria for the target clinical inferencing model based on the distinguishing features. At 820, the multimodal training data generation module 108 can thereafter employ the updated cohort policy criteria for continued training data cohort generation and model optimization.

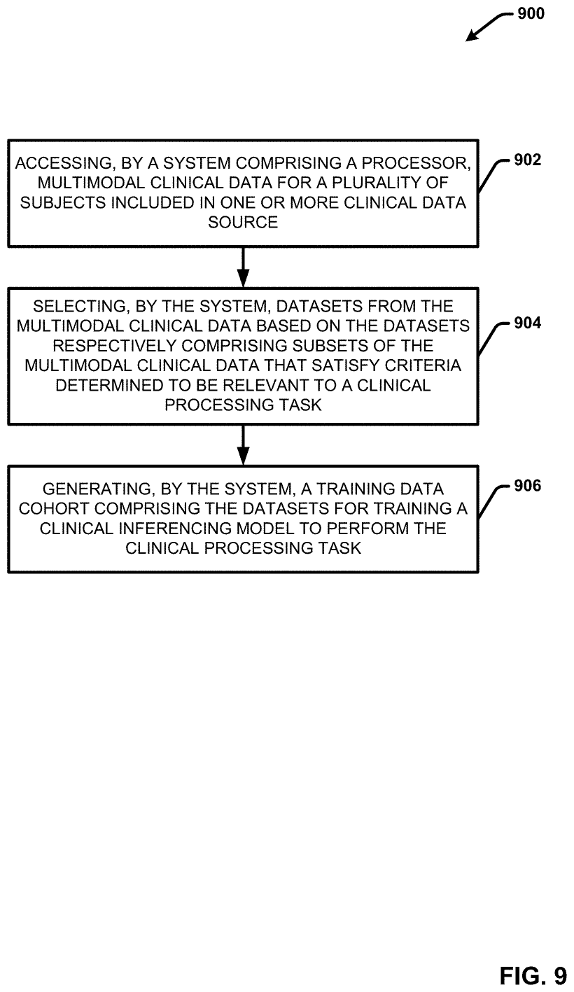

FIG. 9 presents a high-level flow diagram of another example process 900 for generating multimodal training data cohorts tailored to specific clinical ML model inferencing tasks in accordance with one or more embodiments of the disclosed subject matter. Repetitive description of like elements employed in respective embodiments is omitted for sake of brevity.

In accordance with process 900, at 902, a system comprising a processor (e.g., system 100) can access (e.g., via access component 110) multimodal clinical data (e.g., multimodal clinical data 104) for a plurality of subjects included in one or more clinical data sources. At 904, the system can select datasets (e.g., initial multimodal datasets 106 and/or the training cohort datasets 126) from the multimodal clinical data based on the datasets respectively comprising subsets of the multimodal clinical data that satisfy criteria determined to be relevant to a clinical processing task. At 906, the system can generate (e.g., via cohort curation component 116) a training data cohort comprising the datasets (e.g., training cohort datasets 126) for training a clinical inferencing model (e.g., clinical inferencing model 132) to perform the clinical processing task.

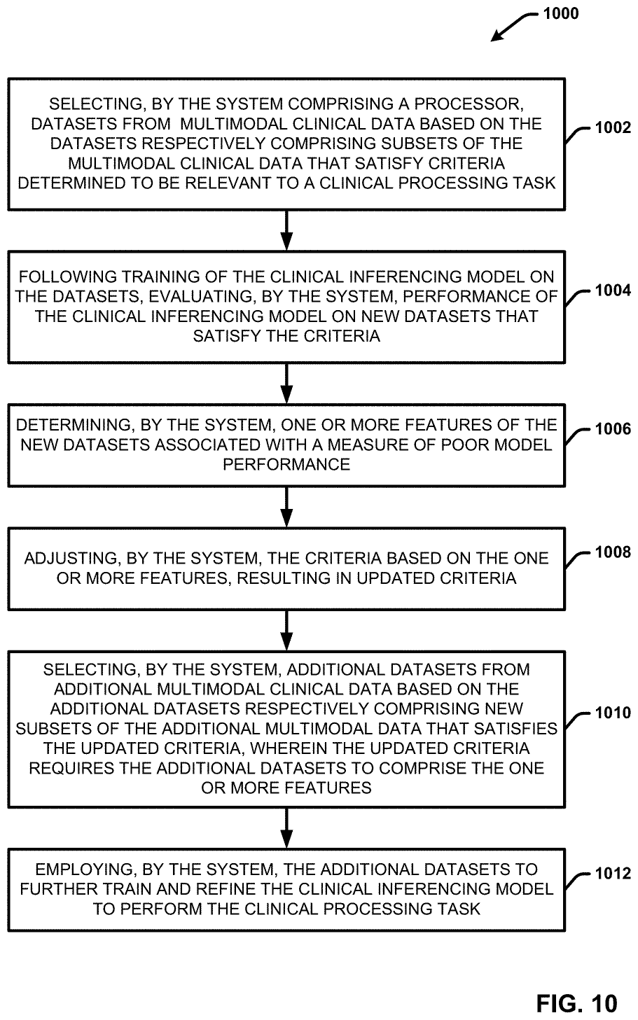

FIG. 10 presents a high-level flow diagram of another example process 1000 for refining multimodal training data cohort selection for a specific clinical ML model in accordance with one or more embodiments of the disclosed subject matter. Repetitive description of like elements employed in respective embodiments is omitted for sake of brevity.

In accordance with process 1000, at 1002, a system comprising a processor (e.g., system 100) can select (e.g., via selection component 112) datasets (e.g., training cohort datasets 126) from multimodal clinical data (e.g., multimodal clinical data 104) based on the datasets respectively comprising subsets of the multimodal clinical data that satisfy criteria determined to be relevant to a clinical processing task. At 1004, following training of the clinical inferencing model on the datasets, the system can evaluate (e.g., via learning component 124) the performance of the clinical inferencing model on new datasets that satisfy the criteria (e.g., new multimodal datasets 136). At 1006, the system can determine one or more features of the new datasets associated with a measure of poor model performance (e.g., via learning component 124). At 1008, the system can adjust the criteria based on the one or more features, resulting in updated criteria (e.g., an updated cohort selection policy for the training data cohort). At 1010, the system can select additional datasets from additional multimodal clinical data (e.g., as new data is added to the multimodal clinical data 104) based on the additional datasets respectively comprising new subsets of the additional multimodal data that satisfied the updated criteria, wherein the updated criteria requires the additional datasets to comprise the one or more features. At 1012, the system can employ the additional datasets to further train and refine the clinical inferencing model to perform the clinical processing task (e.g., with greater accuracy, confidence and/or specificity toward the new additional datasets).

### EXAMPLE OPERATING ENVIRONMENT

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 11, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 11, an example environment 1100 for implementing various aspects of the claimed subject matter includes a computer 1102. The computer 1102 includes a processing unit 1104, a system memory 1106, a codec 1135, and a system bus 1108. The system bus 1108 couples system components including, but not limited to, the system memory 1106 to the processing unit 1104. The processing unit 1104 can be any of various available processors. Dual microprocessors and other multiprocessor architectures also can be employed as the processing unit 1104.

The system bus 1108 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1106 includes volatile memory 1110 and non-volatile memory 1112, which can employ one or more of the disclosed memory architectures, in various embodiments. The basic input/output system (BIOS), containing the basic routines to transfer information between elements within the computer 1102, such as during start-up, is stored in non-volatile memory 1112. In addition, according to present innovations, codec 1135 can include at least one of an encoder or decoder, wherein the at least one of an encoder or decoder can consist of hardware, software, or a combination of hardware and software. Although, codec 1135 is depicted as a separate component, codec 1135 can be contained within non-volatile memory 1112. By way of illustration, and not limitation, non-volatile memory 1112 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1112 can employ one or more of the disclosed memory devices, in at least some embodiments. Moreover, non-volatile memory 1112 can be computer memory (e.g., physically integrated with computer 1102 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1110 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1102 can also include removable/non-removable, volatile/non-volatile computer storage medium. FIG. 11 illustrates, for example, disk storage 1114. Disk storage 1114 includes, but is not limited to, devices like a magnetic disk drive, solid state disk (SSD), flash memory card, or memory stick. In addition, disk storage 1114 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1114 to the system bus 1108, a removable or non-removable interface is typically used, such as interface 1116. It is appreciated that disk storage 1114 can store information related to a user. Such information might be stored at or provided to a server or to an application running on a user device. In one embodiment, the user can be notified (e.g., by way of output device(s) 1136) of the types of information that are stored to disk storage 1114 or transmitted to the server or application. The user can be provided the opportunity to opt-in or opt-out of having such information collected or shared with the server or application (e.g., by way of input from input device(s) 1128).

It is to be appreciated that FIG. 11 describes software that acts as an intermediary between users and the basic computer resources described in the suitable operating environment 1100. Such software includes an operating system 1118. Operating system 1118, which can be stored on disk storage 1114, acts to control and allocate resources of the computer 1102. Applications 1120 take advantage of the management of resources by operating system 1118 through program modules 1124, and program data 1126, such as the boot/shutdown transaction table and the like, stored either in system memory 1106 or on disk storage 1114. It is to be appreciated that the claimed subject matter can be implemented with various operating systems or combinations of operating systems.

A user enters commands or information into the computer 1102 through input device(s) 1128. Input devices 1128 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1104 through the system bus 1108 via interface port(s) 1130. Interface port(s) 1130 include, for example, a serial port, a parallel port, a game port, and a universal serial bus (USB). Output device(s) 1136 use some of the same type of ports as input device(s) 1128. Thus, for example, a USB port can be used to provide input to computer 1102 and to output information from computer 1102 to an output device 1136. Output adapter 1134 is provided to illustrate that there are some output devices 1136 like monitors, speakers, and printers, among other output devices 1136, which require special adapters. The output adapters 1134 include, by way of illustration and not limitation, video and sound cards that provide a means of connection between the output device 1136 and the system bus 1108. It should be noted that other devices or systems of devices provide both input and output capabilities such as remote computer(s) 1138.

Computer 1102 can operate in a networked environment using logical connections to one or more remote computers, such as remote computer(s) 1138. The remote computer(s) 1138 can be a personal computer, a server, a router, a network PC, a workstation, a microprocessor based appliance, a peer device, a smart phone, a tablet, or other network node, and typically includes many of the elements described relative to computer 1102. For purposes of brevity, only a memory storage device 1140 is illustrated with remote computer(s) 1138. Remote computer(s) 1138 is logically connected to computer 1102 through a network interface 1142 and then connected via communication connection(s) 1144. Network interface 1142 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1144 refers to the hardware/software employed to connect the network interface 1142 to the bus 1108. While communication connection 1144 is shown for illustrative clarity inside computer 1102, it can also be external to computer 1102. The hardware/software necessary for connection to the network interface 1142 includes, for exemplary purposes only, internal and external technologies such as, modems including regular telephone grade modems, cable modems and DSL modems, ISDN adapters, and wired and wireless Ethernet cards, hubs, and routers.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
accessing, by a system comprising a processor, multimodal clinical data for a plurality of subjects included in one or more clinical data sources;
selecting, by the system, datasets from the multimodal clinical data based on the datasets respectively comprising subsets of the multimodal clinical data that satisfy criteria determined to be relevant to a clinical processing task; and
generating, by the system, a training data cohort comprising the datasets for training a clinical inferencing model to perform the clinical processing task.

2. The method of claim 1, wherein the multimodal clinical data comprises sets of different types of clinical data for each subject of the plurality of subjects, wherein the subsets respectively comprise clinical data for a different subject of the plurality of subjects, and wherein the selecting comprises selecting the subsets from the sets based on mutual one or more similarity metrics from the different types of clinical data reflecting a consistent anatomy, pathology or diagnosis.

3. The method of claim 1, wherein the criteria varies for different clinical processing tasks and wherein the method further comprises:
determining, by the system, at least some of the criteria using one or more machine learning techniques.

4. The method of claim 1, wherein the selecting comprises:
extracting, by the system, diverse features from the multimodal clinical data;
evaluating, by the system, the diverse features to identify the subsets based on the subsets comprising features of the diverse features that satisfy the criteria; and
importing, by the system, the datasets from the one or more clinical data sources based on the subsets comprising the features.

5. The method of claim 1, wherein the datasets comprise medical images and wherein the generating further comprises:
processing, by the system, the medical images using one or more pre-processing tasks selected from the group consisting of: image harmonization, image style transfer, image resolution augmentation, image data homogenization, and image geometric alignment.

6. The method of claim 1, wherein the criteria comprises first criteria and wherein the selecting comprises:
extracting, by the system, initial datasets from the multimodal data based on the initial datasets respectively comprising initial multimodal data that satisfies the first criteria.

7. The method of claim 6, wherein the criteria further comprises second criteria for features of the initial multimodal data, and wherein the selecting further comprises:
evaluating, by the system, the initial datasets based on the second criteria; and
selecting, by the system, the datasets from the initial datasets based on the multimodal data of the datasets respectively satisfying the second criteria.

8. The method of claim 7, wherein the multimodal data comprises medical images, wherein the second criteria comprises a geometric similarity criterion for the medical images, and wherein the selecting comprises:
determining, by the system, a measure of geometric similarity between the medical images; and
excluding, by the system, images of the medical images from the datasets based on the measure of geometric similarity between the images failing to satisfy a threshold level of geometric similarity.

9. The method of claim 7, the second criteria comprises a biological similarity criterion for the multimodal data, and wherein the selecting comprises:
determining, by the system, a measure of biological similarity between the initial datasets; and
removing, by the system, outlier initial datasets from the datasets based on the measure of biological similarity associated with the outlier initial data sets failing to satisfy a threshold level of biological similarity.

10. The method of claim 1, further comprising:
training, by the system, the clinical inferencing model to perform the clinical processing task using the training data cohort, resulting in a trained clinical inferencing model;
evaluating, by the system, performance of the trained clinical inferencing model on new datasets that satisfy the criteria;
determining, by the system, one or more features of the new datasets associated with a measure of poor model performance; and
adjusting, by the system, the criteria based on the one or more features, resulting in updated criteria.

11. The method of claim of claim 10, further comprising:
receiving, by the system, additional multimodal clinical data for the subjects or new subjects;
selecting, by the system, additional datasets from the additional multimodal clinical data based on the additional datasets respectively comprising new subsets of the additional multimodal data that satisfies the updated criteria, wherein the updated criteria requires the additional datasets to comprise the one or more features;
generating, by the system, a new training data cohort comprising the additional datasets; and
employing, by the system, the new training data cohort to further train and refine the trained clinical inferencing model to perform the clinical processing task.

12. A system, comprising:
a memory that stores computer executable components; and
a processor that executes the computer executable components stored in the memory, wherein the computer executable components comprise:
an access component that accesses multimodal clinical data for a plurality of subjects included in one or more clinical data sources;
a selection component that selects datasets from the multimodal clinical data based on the datasets respectively comprising subsets of the multimodal clinical data that satisfy criteria determined to be relevant to a clinical processing task; and
a cohort curation component that generates a training data cohort comprising the datasets for training a clinical inferencing model to perform the clinical processing task.

13. The system of claim 12, wherein the multimodal clinical data comprises sets of different types of clinical data for each subject of the plurality of subjects, wherein the subsets respectively comprise clinical data for a different subject of the plurality of subjects, and wherein the selecting comprises selecting the subsets from the sets based one or more similarity metrics from the different types of clinical data reflecting a consistent anatomy, pathology, or diagnosis.

14. The system of claim 12, wherein the criteria varies for different clinical processing tasks and wherein the computer executable components further comprise:
a machine learning component that determines at least some of the criteria using one or more machine learning techniques.

15. The system of claim 12, wherein the computer executable components further comprise:
a feature extraction component that extracts diverse features from the multimodal clinical data, wherein the selection component evaluates the diverse features to identify the subsets comprising features of the diverse features that satisfy the criteria; and
an importing component that imports the datasets from the one or more clinical data sources based on the subsets comprising the diverse features.

16. The system of claim 12, wherein the datasets comprise medical images and wherein the computer executable components further comprise:
an image processing that processes the medical images using one or more pre-processing tasks selected from the group consisting of: image harmonization, image style transfer, image resolution augmentation, image data homogenization, and image geometric alignment.

17. The system of claim 12, wherein the criteria comprises first criteria and second criteria, and wherein the computer executable components further comprise:
an extraction component that extracts initial datasets from the multimodal data based on the initial datasets respectively comprising initial multimodal data that that satisfies the first criteria, wherein the selection selects the datasets from the initial datasets based on the multimodal data of the datasets respectively satisfying the second criteria.

18. The system of claim 12, wherein the computer executable components further comprise:
a training component that trains the clinical inferencing model to perform the clinical processing task using the training data cohort, resulting in a trained clinical inferencing model;
a learning component that evaluates performance of the trained clinical inferencing model on new datasets that satisfy the criteria following the training of the clinical inferencing model on the training data cohort and determines one or more features of the new datasets associated with a measure of poor model performance.

19. The system of claim 18, wherein the computer executable components further comprise:
a cohort optimization component that adjusts the criteria based on the one or more features, resulting in updated criteria,
wherein the selection component further selects additional datasets from additional multimodal clinical data based on the additional datasets respectively comprising new subsets of the additional multimodal data that satisfies the updated criteria, wherein the updated criteria requires the new subset to comprise the one or more features,
wherein the curation component further generates a new training data cohort comprising the additional datasets, and
wherein the system employs the new training data cohort to further train and refine the trained clinical inferencing model to perform the clinical processing task.

20. A machine-readable storage medium, comprising executable instructions that, when executed by a processor, facilitate performance of operations, comprising:
accessing multimodal clinical data for a plurality of subjects included in one or more clinical data sources;
selecting datasets from the multimodal clinical data based on the datasets respectively comprising subsets of the multimodal clinical data that satisfy criteria determined to be relevant to a clinical processing task; and
generating a training data cohort comprising the datasets for training a clinical inferencing model to perform the clinical processing task.

21. The machine-readable storage medium of claim 20, wherein the operations further comprise, following the training of the clinical inferencing model on the training data cohort:
evaluating performance of the clinical inferencing model on new datasets that satisfy the criteria;
determining one or more features of the new datasets associated with a measure of poor model performance;
adjusting the criteria based on the one or more features, resulting in updated criteria;
selecting additional datasets from additional multimodal clinical data based on the additional datasets respectively comprising new subsets of the additional multimodal data that satisfies the updated criteria, wherein the updated criteria requires the additional datasets to comprise the one or more features; and
employing the additional datasets to further train and refine the clinical inferencing model to perform the clinical processing task.
